(19) United States
(12) Patent Application Publication
(10) Pub. No.: US 2025/0251323 A1
(43) Pub. Date: Aug. 7, 2025

(54) COLLECTION PROBE AND METHODS OF USE THEREOF

(71) Applicants: BOARD OF REGENTS, THE UNIVERSITY OF TEXAS SYSTEM, Austin, TX (US); BAYLOR COLLEGE OF MEDICINE, Houston, TX (US)

(72) Inventors: Livia Schiavinato EBERLIN, Austin, TX (US); Sunil Prasad BADAL, Austin, TX (US); Grace Yinlei JIANG, Sugar Land, TX (US); Thomas MILNER, Irvine, CA (US); Abigail Nora GATMAITAN, Austin, TX (US); Charles Allen WOLFE, Houston, TX (US)

(21) Appl. No.: 18/856,151

(22) PCT Filed: Apr. 12, 2023
(86) PCT No.: PCT/US2023/018302

§ 371 Date: Oct. 11, 2024

Related U.S. Application Data
(60) Provisional application No. 63/330,886, filed on Apr. 14, 2022.

(51) Int. Cl. G01N 1/38; A61B 10/00; B01L 3/00

(52) U.S. Cl. CPC G01N 1/38; A61B 10/00; B01L 3/502715; B01L 2200/16; B01L 2400/0487; G01N 2001/387

(57) ABSTRACT

Disclosed herein are collection probes, devices/apparatuses comprising the same, and methods of use thereof. The apparatuses can comprise: a probe comprising a reservoir, a first conduit, a second conduit, and a third conduit; wherein the reservoir is in fluid communication with the first, second, and third conduits; and wherein the first conduit is configured to deliver a discrete volume of a solvent from a chamber to the reservoir; the second conduit is configured to deliver a gas from a gas supply to the reservoir; and the third conduit is in fluid communication with a receptacle, the receptacle being configured to receive a sample from the reservoir. Also disclosed herein are comprising: contacting the probe with a surface; applying a fixed or discrete volume of the solvent to the surface; collecting the applied solvent to obtain a liquid sample; and storing the sample in the receptacle.

### CROSS-REFERENCE TO RELATED APPLICATIONS

This application claims the benefit of priority to U.S. Provisional Application No. 63/330,886 filed Apr. 14, 2022, which is hereby incorporated herein by reference in its entirety.

### STATEMENT OF GOVERNMENT SUPPORT

This invention was made with government support under Grant No. R33 CA229068 awarded by the National Institutes of Health. The government has certain rights in this invention.

### BACKGROUND

Devices and methodologies providing for the collection of samples from a variety of sources for subsequent effective molecular assessment are needed for a variety of applications, from clinical diagnosis of diseases to opioid screening. The devices and methods discussed herein address these and other needs.

### SUMMARY

In accordance with the purposes of the disclosed devices, methods, and systems as embodied and broadly described herein, the disclosed subject matter relates to are collection probes, devices or apparatuses comprising the same, and methods of use thereof.

For example, disclosed herein are apparatuses for producing a sample for analysis, the apparatuses comprising: a probe comprising a reservoir, a first conduit, a second conduit, and a third conduit; wherein the reservoir is in fluid communication with the first conduit, the second conduit, and the third conduit; and wherein, when the probe is assembled together with a chamber configured to contain a solvent, a gas supply, and a receptacle, then: the first conduit is configured to be in fluid communication with the chamber, such that the first conduit is configured to deliver a discrete volume of the solvent to the reservoir; the second conduit is configured to be in fluid communication with the gas supply, such that the gas supply is configured to deliver a gas to the reservoir; and the third conduit is in fluid communication with the receptacle, such that the receptacle is configured to receive the sample from the reservoir.

Also disclosed herein are apparatuses for producing a sample for analysis, the apparatuses comprising: a chamber configured to contain a solvent; a gas supply; a receptacle; and a probe comprising a reservoir, a first conduit, a second conduit, and a third conduit; wherein: the reservoir is in fluid communication with the first conduit, the second conduit, and the third conduit; the first conduit is in fluid communication with the chamber, such that the first conduit is configured to deliver a discrete volume of the solvent to the reservoir; the second conduit is in fluid communication with the gas supply and the gas supply is configured to deliver a gas to the reservoir; and the third conduit is in fluid communication with the receptacle and the receptacle is configured to receive the sample from the reservoir.

In some examples, the apparatus further comprises the solvent contained within the chamber. In some examples, the solvent comprises water, an alcohol (e.g., ethanol, methanol), acetonitrile, DMF, or a combination thereof. In some examples, the solvent comprises water, an alcohol (e.g., ethanol, methanol), or a combination thereof. In some examples, the solvent comprises water. In some examples, the solvent comprises ethanol. In some examples, the solvent comprises an aqueous solution. In some examples, the solvent comprises an aqueous mixture of ethanol (e.g., a mixture comprising water and ethanol), wherein the aqueous mixture comprises from 1 to 99%, from 1 to 75%, from 1 to 50%, or from 1 to 25% ethanol. In some examples, the solvent consists essentially of water. In some examples, the solvent consists of water. In some examples, the solvent is sterile. In some examples, the solvent is a pharmaceutically acceptable formulation.

In some examples, the gas comprises air, nitrogen, argon, carbon dioxide, or a combination thereof. In some examples, the gas supply is configured to provide the gas to the reservoir at a pressure of 100 psig or less. In some examples, the gas supply is configured to provide the gas to the reservoir at a pressure of from 0.1 psig to 5.0 psig or from 0.5 psig to 2.5 psig. In some examples, the gas supply is configured to provide the gas at atmospheric pressure. In some examples, the gas supply comprises the atmosphere around the apparatus. In some examples, the gas supply is a pressurized gas supply.

In some examples, the probe is formed from a composition comprising polydimethylsiloxane (PDMS), polytetrafluoroethylene (PTFE), SLA 3D printed elastomer, or a combination thereof. In some examples, the probe is disposable. In some examples, the probe comprises a collection tip that is ejectable.

In some examples, the reservoir is a space formed in the first conduit, the second conduit, the third conduit, or a combination thereof. In some examples, the reservoir is a space formed in said first conduit. In some examples, the reservoir is configured to form and hold a droplet of the solvent. In some examples, the reservoir has a volume of from 0.01 microliters to 500 microliters.

In some examples, the apparatus further comprises a pump in fluid communication with the chamber and the first conduit, wherein the pump is configured to transfer the discrete volume of the solvent from the chamber to the reservoir via the first conduit.

In some examples, the discrete volume is from 0.01 microliters to 500 microliters. In some examples, the discrete volume of the solvent is in direct contact with a surface, the surface being a sample site (e.g., an assay site). In some examples, the solvent is delivered to the reservoir such that it contacts the sample site non-destructively. In some examples, the solvent is delivered to the reservoir and/or contacts the sample site at a pressure of 100 psig or less. In some examples, the solvent is delivered to the reservoir and/or contacts the sample site at a pressure of from 0.1 psig to 5.0 psig or from 0.5 psig to 2.5 psig. In some examples, the surface is at least a portion of a tissue from a subject. In some examples, the tissue is in vivo (e.g., living tissue) or ex vivo.

In some examples, the apparatus further comprises a first valve configured to control a flow from the third conduit to the receptacle. In some examples, the third conduit is under a vacuum when the first valve is in the open position. In some examples, the apparatus further comprises a second valve configured to control a flow of gas through the second conduit.

In some examples, the apparatus further comprises a pump in fluid communication with the third conduit. In some examples, the pump is configured to increase the velocity of the contents within the third conduit.

In some examples, the apparatus further comprises a fourth conduit in fluid communication with the receptacle. In some examples, the apparatus further comprises a pump in fluid communication with the fourth conduit. In some examples, the pump is configured to increase the velocity of the contents within the third conduit.

In some examples, the apparatus further comprises a waste container in fluid communication with the third conduit. In some examples, the apparatus further comprises a valve configured to diverge a fluid from the third conduit to the waste container. In some examples, the apparatus further comprises a pump configured to remove contents of the waste container.

In some examples, the apparatus further comprises a flow constrictor coupled to and/or in fluid communication with at least a portion of the first conduit.

In some examples, the probe and/or the apparatus is configured to be hand-held. In some examples, the apparatus further comprises a housing. In some examples, the probe is disposed within the housing. In some examples, the chamber, gas supply, and probe are disposed within the housing. In some examples, the housing is configured to be hand-held. In some examples, the housing is configured to be disposed within or coupled to a surgical instrument. In some examples, the housing is configured to be disposed within an annulus of a surgical instrument. In some examples, the surgical instrument is a laparoscope, trocar needle, biopsy guide, multiple-lumen catheter, robot, or a combination thereof.

In some examples, the apparatus further comprises a control system configured to control: a solvent flow from the chamber through the first conduit to the reservoir; a gas flow from the gas supply through the second conduit to the reservoir; a sample flow from the reservoir through the third conduit to the receptacle; or a combination thereof. In some examples, the control system is configured to: control the solvent flow at a flow rate of from 100 to 5000 microliters per minute for a period of time of from 1 microsecond to 1 day; control the gas flow at a pressure of from 0 to 100 psig for a period of time of from 1 microsecond to 1 minute; control the sample flow for a period of time of from 1 microsecond to 1 minute; or a combination thereof.

In some examples, the control system comprises a haptic control device (e.g., a switch, a pedal, a button, a knob, a lever, a toggle, etc.) that controls solvent flow (e.g., starts and/or stops).

In some examples, the apparatus further comprises a cart.

In some examples, the apparatus is not directly coupled to an analyzer (e.g., mass spectrometer).

Also disclosed herein are methods of use of any of the apparatuses disclosed herein. For example, also disclosed herein are methods for collecting a sample from a surface using any of the apparatuses disclosed herein, the method comprising: contacting the probe with the surface; applying a fixed or discrete volume of the solvent to the surface; collecting the applied solvent to obtain a liquid sample; and storing the sample in the receptacle.

In some examples, the fixed or discrete volume of a solvent is not applied as a spray. In some examples, the fixed or discrete volume of a solvent is applied as a droplet. In some examples, the fixed or discrete volume of a solvent is applied using a pressure of 100 psig or less. In some examples, the fixed or discrete volume of a solvent is applied using a pressure of 10 psig or less. In some examples, the apparatus includes the flow constrictor and the fixed or discrete volume of solvent is applied using the flow constrictor.

In some examples, collecting the applied solvent comprises applying a negative pressure to pull the sample into the third conduit and/or applying a gas pressure to push the sample into the third conduit and then into the receptacle. In some examples, collecting the applied solvent comprises applying a negative pressure to pull the sample into the third conduit and applying a positive pressure to push the sample into the third conduit and then into the receptacle.

In some examples, the solvent is applied through the first conduit that is separate from the third conduit. In some examples, the gas pressure is applied through the second conduit that is separate from the first conduit and the third conduit.

In some examples, applying a gas pressure to push the sample into the third conduit comprises applying a pressure of 100 psig or less.

In some examples, the surface comprises at least a portion of a tissue of a subject, e.g. such that the sample site is a tissue site. In some examples, the method produces no detectable physical damage to the tissue. In some examples, the tissue site in an internal tissue site that is being surgically assessed. In some examples, the method does not involve application of ultrasonic or vibrational energy to the tissue.

In some examples, the discrete volume of solvent from 0.01 to 500 μL. In some examples, the discrete volume of solvent is from 0.1 to 150 μL, from 0.1 to 100 μL, or from 1 and 50 μL.

In some examples, the solvent is contacted with the surface for an amount of time of from 1 microsecond to 1 day before the liquid sample is collected. In some examples, the solvent is contacted with the surface for an amount of time of from 0.1 seconds to 1 hour, from 0.1 seconds to 1 minute, from 0.1 seconds to 30 seconds, or from 1 second to 10 seconds before the liquid sample is collected. In some examples, the solvent is contacted with the surface for an amount of time of from 1 second to 5 seconds before the liquid sample is collected.

In some examples, the method comprises collecting a plurality of liquid samples from a plurality of sites. In some examples, the method further comprises washing the probe between collection of the different samples. In some examples, the probe is disposable and is changed between collection of the different samples. In some examples, the probe comprises a collection tip and further comprising ejecting the collection tip from the probe after the liquid samples are collected. In some examples, the method further comprises replacing the receptacle with an empty receptacle between collection of the different samples. In some examples, the plurality of sites comprises 2 or more, 3 or more, 4 or more, 5 or more, 6 or more, 7 or more, 8 or more, 9 or more, or 10 or more sites. In some examples, the plurality of tissue sites surrounds a section of tissue that has been surgically resected. In some examples, the resected tissue is a tumor.

In some examples, the method is further defined as an intraoperative method. In some examples, the method is in vivo or ex vivo.

In some examples, the method further comprises removing the receptacle (containing the sample) from the apparatus and transporting the receptacle containing the sample to an analyzer. In some examples, the method further comprises subjecting the sample to analysis to determine a property of the sample and/or site. In some examples, the analysis comprises mass spectrometry analysis. In some examples, the mass spectrometry analysis comprises determining a profile corresponding to the site. In some examples, the method further comprises comparing the profile to a reference profile to determine a property of the sample and/or the sample site.

In some examples, the property comprises the presence or absence of an analyte of interest in the sample; the concentration of an analyte of interest in the sample; the identity of the analyte of interest in the sample; or a combination thereof.

In some examples, the analyte of interest is a biomarker. In some examples, the biomarker is indicative of a disease. In some examples, the method further comprises diagnosing and/or monitoring a disease in a subject based on the property of the sample. In some examples, the disease comprises endometriosis. In some examples, the disease comprises cancer. In some examples, the method comprises identifying tissue sites that include diseased tissue. In some examples, the diseased tissues comprise cancer cells. In some examples, the diseased tissues comprise lung, ovarian, thyroid, or breast cancer cells. In some examples, the method further comprises selecting a course of treatment for the disease. In some examples, the method further comprises resecting tissue sites that are identified to include diseased tissue. In some examples, the method further comprises administering an anti-cancer therapy to the subject.

In some examples, the analyte of interest comprises a chemical, such as a medicament or illegal substance. In some examples, the analyte of interest comprises an opioid.

Additional advantages of the disclosed devices, systems, and methods will be set forth in part in the description which follows, and in part will be obvious from the description. The advantages of the disclosed devices, systems, and methods will be realized and attained by means of the elements and combinations particularly pointed out in the appended claims. It is to be understood that both the foregoing general description and the following detailed description are exemplary and explanatory only and are not restrictive of the disclosed devices, systems, and methods, as claimed.

The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

The accompanying figures, which are incorporated in and constitute a part of this specification, illustrate several aspects of the disclosure, and together with the description, serve to explain the principles of the disclosure.

FIG. 1. Schematic representation of the MasSpec Pen system. The pen-sized handheld device is directly integrated into a sampling interface through PTFE tubing (or another highly hydrophobic material). The interface houses the pinch valves, microcontroller, and tubing to connect the system to the receptacle. The system is automatically triggered by the user through a foot pedal.

FIG. 2. The MasSpec Pen is designed with a PDMS 3D-printed tip and three PTFE conduits, which provide incoming water to the tip, gas, and an outgoing conduit for the water droplet.

FIG. 3. Schematic representation of the MasSpec Pen system and operation steps. The tip contacts the tissue for analysis, and it is designed with 3 conduits and a solvent reservoir. When the system is triggered (t=0 sec) by the use through the pedal, the syringe pump delivers a controlled volume of water to the reservoir. The discrete water droplet interacts with the tissue to extract molecules. After, in this case, 3 seconds of extraction, the vacuum conduit is opened to transport the droplet from the MasSpec Pen to the receptacle through the tubing system for collection.

FIG. 4. Schematic representation of the MasSpec Pen system according to one example implementation.

FIG. 5. Show enlarged views of the tip of an example MasSpec Pen according to one implementation.

FIG. 6. Show enlarged views of the tip of an example MasSpec Pen according to one implementation.

FIG. 7. Schematic representation of an alternative configuration of a system of the embodiments

FIG. 8. Schematic representation of an alternative configuration of a system of the embodiments.

FIG. 9. A schematic illustration of an example computing device.

FIG. 10. Multilumen tubing for use with the probe for minimally invasive surgery.

FIG. 11. A cannula and trocar needle for housing and inserting the probe for minimally invasive surgery.

FIG. 12. The handheld MasSpec Pen contains a PDMS tip and three PTFE conduits, which provide incoming water (1) and gas (2) to the tip, and an outgoing conduit (3) for the water droplet to the mass spectrometer. The pen tip holds a water droplet within the reservoir, which contacts tissue for analysis.

FIG. 13. Schematic representation of modular MasSpec Pen system for sample collection.

FIG. 14. Photograph of modular MasSpec Pen system for sample collection.

FIG. 15. Solvent optimization—best combination of variables.

FIG. 16. Solvent optimization—second best combination of variables.

FIG. 17. Calibration curve and LOD of oxycodone pure standard.

FIG. 18. Calibration curve and LOD of hydrocodone pure standard.

FIG. 19. Calibration curve and LOD of fentanyl pure standard.

FIG. 20. Calibration curves and LOD of oxycodone in a mixture together with hydrocodone and fentanyl.

FIG. 21. Calibration curves and LOD of hydrocodone in a mixture together with oxycodone and fentanyl.

FIG. 22. Calibration curves and LOD of fentanyl in a mixture together with oxycodone and hydrocodone.

FIG. 23. The EIC of oxycodone from the surface of autopsy skin samples for time zero of a skin desorption study.

FIG. 24. The EIC of oxycodone from the surface of autopsy skin samples after 24 hrs of a skin desorption study.

FIG. 25. Schematic of modular MSPen cart system.

FIG. 26. Schematic of modular MSPen cart system.

FIG. 27. Photograph of modular MSPen cart system.

FIG. 28. Photograph of modular MSPen cart system.

FIG. 29. Photograph of modular MSPen cart system.

FIG. 30. Schematic of detached MasSpec pen that stores the sample within the device unit.

FIG. 31. Schematic of detached MasSpec pen that stores the sample within the device unit.

FIG. 32. An enlarged schematic of the three channel elastomer tip.

FIG. 33. An illustration of the operational steps for the fully detached MasSpec Pen.

FIG. 34. Schematic illustration of MasSpec pen with flow constrictor.

FIG. 35. Effect of flow constrictor on results collected for mouse brain extract (MBE) with detached MasSpec Pen (DMSP). The use of the flow constrictor increased the lipid signal (bottom trace).

FIG. 36. LC-MS results for mouse brain extract (MBE) collected with detached MasSpec Pen (DMSP).

### DETAILED DESCRIPTION

The devices, methods, and systems described herein may be understood more readily by reference to the following detailed description of specific aspects of the disclosed subject matter and the Examples included therein.

Before the present devices, methods, and systems are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings.

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

“Exemplary” means “an example of” and is not intended to convey an indication of a preferred or ideal embodiment. “Such as” is not used in a restrictive sense, but for explanatory purposes.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

As used herein the term “plurality” means 2 or more (e.g., 3 or more; 4 or more; 5 or more; 10 or more; 15 or more; 20 or more; 25 or more; 30 or more; 40 or more; 50 or more; 75 or more; 100 or more; 150 or more; 200 or more; 250 or more; 300 or more; 400 or more; 500 or more; 750 or more; 1000 or more; 1500 or more; 2000 or more; 2500 or more; 3000 or more; 4000 or more; or 5000 or more).

As used herein, “sample” or “liquid samples” can refer to extracts from tissues or other biological specimens (e.g., extracts comprising proteins and metabolites) obtained by contacting tissue or biological specimen with a solvent according to the embodiments. In some examples, a sample can be an extract from a non-biological specimen, such as the surface on an object (e.g., a forensic sample).

As used herein, “essentially free,” in terms of a specified component, is used herein to mean that none of the specified components has been purposefully formulated into a composition and/or is present only as a contaminant or in trace amounts. The total amount of the specified component resulting from any unintended contamination of a composition is therefore well below 0.01%. Most preferred is a composition in which no amount of the specified component can be detected with standard analytical methods.

As used herein in the specification and claims, the terms “conduit” and “tube” are used interchangeably and refer to a structure that can be used to direct flow of a gas or liquid.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

The term “inhibit” refers to a decrease in an activity, response, condition, disease, or other biological parameter. This can include but is not limited to the complete ablation of the activity, response, condition, or disease. This can also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “artificial intelligence” is defined herein to include any technique that enables one or more computing devices or comping systems (i.e., a machine) to mimic human intelligence. Artificial intelligence (AI) includes, but is not limited to, knowledge bases, back- propagation bases, machine learning, representation learning, and deep learning.

The term “machine learning” is defined herein to be a subset of AI that enables a machine to acquire knowledge by extracting patterns from raw data. Machine learning techniques include, but are not limited to, logistic regression, support vector machines (SVMs), decision trees, Naïve Bayes classifiers, and artificial neural networks. The term “representation learning” is defined herein to be a subset of machine learning that enables a machine to automatically discover representations needed for feature detection, prediction, or classification from raw data. Representation learning techniques include, but are not limited to, autoencoders. The term “deep learning” is defined herein to be a subset of machine learning that that enables a machine to automatically discover representations needed for feature detection, prediction, classification, etc. using layers of processing. Deep learning techniques include, but are not limited to, artificial neural network or multilayer perceptron (MLP).

Machine learning models include supervised, semi-supervised, and unsupervised learning models. In a supervised learning model, the model learns a function that maps an input (also known as feature or features) to an output (also known as target or target) during training with a labeled data set (or dataset). In an unsupervised learning model, the model learns a function that maps an input (also known as feature or features) to an output (also known as target or target) during training with an unlabeled data set. In a semi-supervised model, the model learns a function that maps an input (also known as feature or features) to an output (also known as target or target) during training with both labeled and unlabeled data.

### Collection Probes and Apparatuses

Disclosed herein are collection probes, devices or apparatuses comprising the same, and methods of use thereof.

For example, disclosed herein are apparatuses for producing a sample (e.g., one or more samples) for analysis, the apparatus comprising: a probe comprising a reservoir, a first conduit, a second conduit, and a third conduit; wherein the reservoir is in fluid communication with the first conduit, the second conduit, and the third conduit.

When the probe is assembled together with a chamber configured to contain a solvent, a gas supply, and a receptacle, then: the first conduit is configured to be in fluid communication with the chamber, such that the first conduit is configured to deliver a discrete volume of the solvent to the reservoir; the second conduit is configured to be in fluid communication with the gas supply, such that the gas supply is configured to deliver a gas to the reservoir; and the third conduit is in fluid communication with the receptacle, such that the receptacle is configured to receive the sample from the reservoir.

In some examples, the apparatus can further comprise the receptacle, the chamber the gas supply, the solvent, or a combination thereof.

For example, also disclosed herein are apparatuses for producing a sample for analysis, the apparatus comprising: a chamber configured to contain a solvent; a gas supply; a receptacle; and a probe comprising a reservoir, a first conduit, a second conduit, and a third conduit; wherein: the reservoir is in fluid communication with the first conduit, the second conduit, and the third conduit; the first (solvent) conduit is in fluid communication with the chamber, such that the first conduit is configured to deliver a discrete volume of the solvent to the reservoir; the second (gas) conduit is in fluid communication with the gas supply and the gas supply is configured to deliver a gas to the reservoir; and the third (collection) conduit is in fluid communication with the receptacle and the receptacle is configured to receive the sample from the reservoir.

The receptacle can, for example, comprise any suitable container for collecting the sample (e.g., a liquid sample), such as those known in the art. In some examples, the receptacle can comprise a vial, a suction canister, a vacutainer, etc.

The receptacle can, for example, comprise any suitable material, such as those known in the art. For example, the receptacle can comprise glass, a polymer, or a combination thereof. For example, the receptacle can comprise a polymer liner disposed inside of a rigid canister. In some examples, the receptacle can be a solid matrix or a solid porous matrix comprised of a material that draws the solvent and molecules in the matrix through surface tension forces. In this embodiment, the interstitial spaces in porous matrix hold the liquid sample and allow transport to a distant location.

In some examples, the receptacle can contain a substance, such a fluid and/or a solid, such that the sample mixes with said substance and/or is absorbed by said substance within the receptacle when the sample reaches the receptacle. The substance can, for example, comprise another solvent, a buffer solution, etc.

The receptacle is a closed container configured to be fluidly coupled to the third conduit. For example, the receptacle can have an interior volume defined by a wall, wherein the wall has one or more ports independently configured to receive the third conduit, such that the interior volume of the receptacle is fluidly connected to the third conduit. In some examples, the receptacle can comprise a body and a lid, wherein the lid is configured to be coupled to the body (e.g., removably coupled), such that when the lid and the body are coupled they define the interior volume. The lid can, for example, have a port, the port being configured to receive the third conduit.

The chamber can, for example, comprise any suitable container for containing a solvent, such as those known in the art. The chamber can, for example, comprise any suitable material, such as those known in the art. For example, the chamber can comprise a polymer, glass, or a combination thereof. In some examples, the chamber can be rigid or flexible. In some examples, the chamber can comprise a sterile medical collection bag, a rigid polymer vessel, a glass vessel, or a combination thereof. In some examples, the chamber can comprise a syringe.

In some examples, the apparatus further comprises the solvent contained within the chamber. The solvent can comprise any suitable solvent, such as those known in the art. The solvent can, for example, comprise tetrahydrofuran (THF), N-methyl-2-pyrrolidone (NMP), dimethylformamide (DMF), N-methylformamide, formamide, dichloromethane (CH₂Cl₂), ethylene glycol, polyethylene glycol, glycerol, alkane diol, ethanol, methanol, propanol, isopropanol, water, acetonitrile, chloroform, toluene, methyl acetate, ethyl acetate, acetone, hexane, heptane, tetraglyme, propylene carbonate, diglyme, dimethyl sulfoxide (DMSO), dimethoxyethane, xylene, dimethylacetamide, or combinations thereof. In some examples, the solvent is a pharmaceutically acceptable formulation. In some examples, the solvent is sterile.

In some examples, the solvent comprises water, an alcohol (e.g., ethanol, methanol), acetonitrile, DMF, or a combination thereof. In some examples, the solvent comprises water, an alcohol (e.g., ethanol, methanol), or a combination thereof. In some examples, the solvent comprises water. In some examples, the solvent consists essentially of water. In some examples, the solvent consists of water.

In some examples, the solvent comprises 1% or more of an alcohol (e.g., ethanol and/or methanol) (e.g., 2% or more, 3% or more, 4% or more, 5% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, or 95% or more). In some examples, the solvent comprises 100% or less of an alcohol (e.g., ethanol and/or methanol) (e.g., 99% or less, 98% or less, 97% or less, 96% or less, 95% or less, 90% or less, 85% or less, 80% or less, 75% or less, 70% or less, 65% or less, 60% or less, 55% or less, 50% or less, 45% or less, 40% or less, 35% or less, 30% or less, 25% or less, 20% or less, 15% or less, 10% or less, or 5% or less). The amount of alcohol in the solvent can range from any of the minimum values described above to any of the maximum values described above. For example, the solvent can comprise from 1% to 100% of an alcohol (e.g., ethanol and/or methanol) (e.g., from 1% to 50%, from 50% to 100%, from 1% to 25%, from 25% to 50%, from 50% to 75%, from 75% to 100%, from 1% to 20%, from 20% to 40%, from 40% to 60%, from 60% to 80%, from 80% to 100%, from 1% to 99%, 1% to 95%, from 1% to 90%, from 1% to 80%, from 1% to 75%, from 1% to 60%, from 1% to 40%, from 1% to 30%, from 2% to 100%, from 5% to 100%, from 10% to 100%, from 15% to 100%, from 20% to 100%, from 25% to 100%, from 30% to 100%, from 40% to 100%, from 60% to 100%, from 90% to 100%, from 2% to 99%, from 5% to 95%, from 10% to 90%, from 20% to 80%, from 30% to 70%, or from 40% to 60%).

In some examples, the solvent comprises ethanol. In some examples, the solvent comprises an aqueous solution. In some examples, the solvent comprises an aqueous mixture of ethanol (e.g., a mixture comprising water and ethanol). In some examples, the aqueous mixture comprises 1% or more of ethanol (e.g., 2% or more, 3% or more, 4% or more, 5% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, or 95% or more). In some examples, the aqueous mixture comprises 99% or less of ethanol (e.g., 98% or less, 97% or less, 96% or less, 95% or less, 90% or less, 85% or less, 80% or less, 75% or less, 70% or less, 65% or less, 60% or less, 55% or less, 50% or less, 45% or less, 40% or less, 35% or less, 30% or less, 25% or less, 20% or less, 15% or less, 10% or less, or 5% or less). The amount of ethanol in the aqueous mixture can range from any of the minimum values described above to any of the maximum values described above. For example, the aqueous mixture can comprise from 1% to 99% of ethanol (e.g., from 1% to 50%, from 50% to 99%, from 1% to 25%, from 25% to 50%, from 50% to 75%, from 75% to 99%, from 1% to 20%, from 20% to 40%, from 40% to 60%, from 60% to 80%, from 80% to 99%, from 1% to 98%, 1% to 95%, from 1% to 90%, from 1% to 80%, from 1% to 75%, from 1% to 60%, from 1% to 40%, from 1% to 30%, from 1% to 10%, from 1% to 5%, from 2% to 99%, from 5% to 99%, from 10% to 99%, from 15% to 99%, from 20% to 99%, from 25% to 99%, from 30% to 99%, from 40% to 99%, from 60% to 99%, from 90% to 99%, from 2% to 98%, from 5% to 95%, from 10% to 90%, from 20% to 80%, from 30% to 70%, or from 40% to 60%).

The gas can comprise any suitable gas. In some examples, the gas comprises air, nitrogen, argon, carbon dioxide, or a combination thereof. In some examples, the gas comprises air. In some examples, the gas comprises an inert gas, such as nitrogen or argon. In some examples the gas comprises carbon dioxide.

In some examples, the gas supply is configured to provide the gas to the reservoir at a pressure of 0 psig or more (e.g., 0.1 psig or more, 0.2 psig or more, 0.3 psig or more, 0.4 psig or more, 0.5 psig or more, 0.75 psig or more, 1 psig or more, 1.25 psig or more, 1.5 psig or more, 2 psig or more, 2.5 psig or more, 3 psig or more, 3.5 psig or more, 4 psig or more, 4.5 psig or more, 5 psig or more, 6 psig or more, 7 psig or more, 8 psig or more, 9 psig or more, 10 psig or more, 15 psig or more, 20 psig or more, 25 psig or more, 30 psig or more, 35 psig or more, 40 psig or more, 45 psig or more, 50 psig or more, 55 psig or more, 60 psig or more, 65 psig or more, 70 psig or more, 75 psig or more, 80 psig or more, 85 psig or more, 90 psig or more, or 95 psig or more). In some examples, the gas supply is configured to provide the gas to the reservoir at a pressure of 100 psig or less (e.g., 99 psig or less, 98 psig or less, 97 psig or less, 96 psig or less, 95 psig or less, 90 psig or less, 85 psig or less, 80 psig or less, 75 psig or less, 70 psig or less, 65 psig or less, 60 psig or less, 55 psig or less, 50 psig or less, 45 psig or less, 40 psig or less, 35 psig or less, 30 psig or less, 25 psig or less, 20 psig or less, 15 psig or less, 10 psig or less, 9 psig or less, 8 psig or less, 7 psig or less, 6 psig or less, 5 psig or less, 4.5 psig or less, 4 psig or less, 3.5 psig or less, 3 psig or less, 2.5 psig or less, 2 psig or less, 1.5 psig or less, 1.25 psig or less, 1 psig or less, 0.75 psig or less, 0.5 psig or less, 0.4 psig or less, 0.3 psig or less, 0.2 psig or less, or 0.1 psig or less). The pressure at which the gas supply provides the gas to the reservoir can range from any of the minimum values described above to any of the maximum values described above. For example, the gas supply can be configured to provide the gas to the reservoir at a pressure of from 0 psig to 100 psig (e.g., from 0 psig to 50 psig, from 50 psig to 100 psig, from 0 psig to 25 psig, from 25 psig to 50 psig, from 50 psig to 75 psig, from 75 psig to 100 psig, from 0 psig to 20 psig, from 20 psig to 40 psig, from 40 psig to 60 psig, from 60 psig to 80 psig, from 80 psig to 100 psig, from 0 psig to 99 psig, 0 psig to 95 psig, from 0 psig to 90 psig, from 0 psig to 80 psig, from 0 psig to 75 psig, from 0 psig to 60 psig, from 0 psig to 40 psig, from 0 psig to 30 psig, from 0.1 psig to 100 psig, from 5 psig to 100 psig, from 10 psig to 100 psig, from 15 psig to 100 psig, from 20 psig to 100 psig, from 25 psig to 100 psig, from 30 psig to 100 psig, from 40 psig to 100 psig, from 60 psig to 100 psig, from 90 psig to 100 psig, from 0.1 psig to 99 psig, from 5 psig to 95 psig, from 10 psig to 90 psig, from 20 psig to 80 psig, from 30 psig to 70 psig, from 40 psig to 60 psig, from 0.1 psig to 100 psig, from 0.1 psig to 99 psig, 0.1 psig to 95 psig, from 0.1 psig to 90 psig, from 0.1 psig to 80 psig, from 0.1 psig to 75 psig, from 0.1 psig to 60 psig, from 0.1 psig to 40 psig, from 0.1 psig to 30 psig, from 0.1 psig to 25 psig, from 0.1 psig to 20 psig, from 0.1 psig to 15 psig, from 0.1 psig to 10 psig, from 0.1 psig to 5 psig, or from 0.5 psig to 2.5 psig). As used herein, psig refers to gauge pressure which is defined as the pressure less surrounding atmospheric pressure.

In some examples, the gas supply is configured to provide the gas at atmospheric pressure. In some examples, the gas supply is at atmospheric pressure. For example, the conduit for delivery of a gas can be open to the atmosphere around the apparatus such that the gas is supplied by the atmosphere around the apparatus. For example, the supply can comprise the atmosphere around the apparatus. In some examples, the gas supply is a pressurized gas supply, such as a pressurized gas cylinder or tank. In some examples, the gas can be pumped into the apparatus. Likewise, in some examples, the gas can be pulled through an apparatus by use of a vacuum.

The probe can be formed from (e.g., comprise) any suitable material. In certain examples, the probe can be biocompatible, e.g., the probe can comprise a biocompatible material. The probe can be rigid, flexible, or a combination thereof.

In some examples, the probe comprises a polymer, a composite material, a ceramic, a metal, or a combination thereof. Examples of suitable materials include, but are not limited to, resins, elastomers, alumina, silica, glass, ceramic, titanium dioxide, zirconia, calcium phosphates, and polymers. In some examples, the probe can comprise a non-porous elastomer and/or resin. In some examples, the probe comprises a polymer. The probe can, for example, be formed from a composition comprising polydimethylsiloxane (PDMS), polytetrafluoroethylene (PTFE), SLA 3D printed elastomer, or a combination thereof. In some examples, the probe can be formed from a composition comprising polydimethylsiloxane (PDMS) and/or polytetrafluoroethylene (PTFE). In some examples, the probe can be formed from a composition comprising SLA 3D printed resin and PDMS elastomer.

In some examples, the probe can be disposable. In some examples, the probe can be reusable. In some examples, the probe comprises a collection tip that is ejectable (e.g., capable of being ejected from the probe). In certain examples, the apparatus further includes a means for ejecting the collection tip, e.g. a mechanism that allows the collection to be mechanically separated from the probe.

The reservoir can, for example, be a space formed in the first conduit, the second conduit, the third conduit, or a combination thereof. In some examples, the reservoir is a space formed in said first conduit. The reservoir can, for example, be configured to form and hold a droplet (e.g., a single droplet) of the solvent.

In some examples, the reservoir can have a volume of 0.01 microliters or more (e.g., 0.05 microliters or more, 0.1 microliters or more, 0.5 microliters or more, 0.75 microliters or more, 1 microliters or more, 1.25 microliters or more, 1.5 microliters or more, 2 microliters or more, 2.5 microliters or more, 3 microliters or more, 3.5 microliters or more, 4 microliters or more, 4.5 microliters or more, 5 microliters or more, 6 microliters or more, 7 microliters or more, 8 microliters or more, 9 microliters or more, 10 microliters or more, 15 microliters or more, 20 microliters or more, 25 microliters or more, 30 microliters or more, 35 microliters or more, 40 microliters or more, 45 microliters or more, 50 microliters or more, 60 microliters or more, 70 microliters or more, 80 microliters or more, 90 microliters or more, 100 microliters or more, 125 microliters or more, 150 microliters or more, 175 microliters or more, 200 microliters or more, 225 microliters or more, 250 microliters or more, 300 microliters or more, 350 microliters or more, 400 microliters or more, or 450 microliters or more). In some examples, the reservoir can have a volume of 500 microliters or less (e.g., 450 microliters or less, 400 microliters or less, 350 microliters or less, 300 microliters or less, 250 microliters or less, 225 microliters or less, 175 microliters or less, 150 microliters or less, 125 microliters or less, 100 microliters or less, 90 microliters or less, 80 microliters or less, 70 microliters or less, 60 microliters or less, 50 microliters or less, 45 microliters or less, 40 microliters or less, 35 microliters or less, 30 microliters or less, 25 microliters or less, 20 microliters or less, 15 microliters or less, 10 microliters or less, 9 microliters or less, 8 microliters or less, 7 microliters or less, 6 microliters or less, 5 microliters or less, 4.5 microliters or less, 4 microliters or less, 3.5 microliters or less, 3 microliters or less, 2.5 microliters or less, 2 microliters or less, 1.5 microliters or less, 1.25 microliters or less, 1 microliters or less, 0.75 microliters or less, 0.5 microliters or less, 0.1 microliters or less, or 0.05 microliters or less). The volume of the reservoir can range from any of the minimum values described above to any of the maximum values described above. For example, the reservoir can have a volume of from 0.01 microliters to 500 microliters (e.g., from 0.01 microliters to 250 microliters, from 250 microliters to 500 microliters, from 0.01 microliters to 0.1 microliters, from 0.1 microliters to 1 microliters, from 1 microliters to 10 microliters, from 10 microliters to 100 microliters, from 100 microliters to 500 microliters, from 0.01 microliters to 400 microliters, from 0.01 microliters to 300 microliters, from 0.01 microliters to 200 microliters, from 0.01 microliters to 150 microliters, from 0.01 microliters to 100 microliters, from 0.01 microliters to 50 microliters, from 0.01 microliters to 20 microliters, from 0.01 microliters to 10 microliters, from 0.1 microliters to 500 microliters, from 0.5 microliters to 500 microliters, from 1 microliters to 500 microliters, from 5 microliters to 500 microliters, from 10 microliters to 500 microliters, from 20 microliters to 500 microliters, from 50 microliters to 500 microliters, from 100 microliters to 500 microliters, from 200 microliters to 500 microliters, from 300 microliters to 500 microliters, from 400 microliters to 500 microliters, from 0.1 microliters to 450 microliters, from 0.1 microliters to 400 microliters, from 0.1 microliters to 150 microliters, from 1 microliters to 150 microliters, from 0.1 microliters to 100 microliters, from 1 microliters to 100 microliters, from 2 microliters to 50 microliters, or from 5 microliters to 20 microliters).

The first conduit is configured to deliver a discrete volume of the solvent to the reservoir. In some examples, the apparatus further comprises a pump in fluid communication with the chamber and the first conduit, wherein the pump is configured to transfer the discrete volume of the solvent from the chamber to the reservoir via the first conduit.

The pump can comprise any suitable pump, such as those known in the art. For example, the pump can be a peristaltic pump (e.g., a roller pump), a diaphragm pump, a gear pump, a syringe pump, a piston pump, a rotary pump, or a vacuum pump. In some examples, the pump comprises a syringe pump.

The discrete volume of the solvent can, for example, be 0.01 microliters or more (e.g., 0.05 microliters or more, 0.1 microliters or more, 0.5 microliters or more, 0.75 microliters or more, 1 microliters or more, 1.25 microliters or more, 1.5 microliters or more, 2 microliters or more, 2.5 microliters or more, 3 microliters or more, 3.5 microliters or more, 4 microliters or more, 4.5 microliters or more, 5 microliters or more, 6 microliters or more, 7 microliters or more, 8 microliters or more, 9 microliters or more, 10 microliters or more, 15 microliters or more, 20 microliters or more, 25 microliters or more, 30 microliters or more, 35 microliters or more, 40 microliters or more, 45 microliters or more, 50 microliters or more, 60 microliters or more, 70 microliters or more, 80 microliters or more, 90 microliters or more, 100 microliters or more, 125 microliters or more, 150 microliters or more, 175 microliters or more, 200 microliters or more, 225 microliters or more, 250 microliters or more, 300 microliters or more, 350 microliters or more, 400 microliters or more, or 450 microliters or more). In some examples, discrete volume of the solvent can be 500 microliters or less (e.g., 450 microliters or less, 400 microliters or less, 350 microliters or less, 300 microliters or less, 250 microliters or less, 225 microliters or less, 175 microliters or less, 150 microliters or less, 125 microliters or less, 100 microliters or less, 90 microliters or less, 80 microliters or less, 70 microliters or less, 60 microliters or less, 50 microliters or less, 45 microliters or less, 40 microliters or less, 35 microliters or less, 30 microliters or less, 25 microliters or less, 20 microliters or less, 15 microliters or less, 10 microliters or less, 9 microliters or less, 8 microliters or less, 7 microliters or less, 6 microliters or less, 5 microliters or less, 4.5 microliters or less, 4 microliters or less, 3.5 microliters or less, 3 microliters or less, 2.5 microliters or less, 2 microliters or less, 1.5 microliters or less, 1.25 microliters or less, 1 microliters or less, 0.75 microliters or less, 0.5 microliters or less, 0.1 microliters or less, or 0.05 microliters or less). The discrete volume of the solvent can range from any of the minimum values described above to any of the maximum values described above. For example, the discrete volume of the solvent can be from 0.01 microliters to 500 microliters (e.g., from 0.01 microliters to 250 microliters, from 250 microliters to 500 microliters, from 0.01 microliters to 0.1 microliters, from 0.1 microliters to 1 microliters, from 1 microliters to 10 microliters, from 10 microliters to 100 microliters, from 100 microliters to 500 microliters, from 0.01 microliters to 400 microliters, from 0.01 microliters to 300 microliters, from 0.01 microliters to 200 microliters, from 0.01 microliters to 150 microliters, from 0.01 microliters to 100 microliters, from 0.01 microliters to 50 microliters, from 0.01 microliters to 20 microliters, from 0.01 microliters to 10 microliters, from 0.1 microliters to 500 microliters, from 0.5 microliters to 500 microliters, from 1 microliters to 500 microliters, from 5 microliters to 500 microliters, from 10 microliters to 500 microliters, from 20 microliters to 500 microliters, from 50 microliters to 500 microliters, from 100 microliters to 500 microliters, from 200 microliters to 500 microliters, from 300 microliters to 500 microliters, from 400 microliters to 500 microliters, from 0.1 microliters to 450 microliters, from 0.1 microliters to 400 microliters, from 0.1 microliters to 150 microliters, from 1 microliters to 150 microliters, from 0.1 microliters to 100 microliters, from 1 microliters to 100 microliters, from 2 microliters to 50 microliters, or from 5 microliters to 20 microliters).

In some examples, the discrete volume of the solvent is in direct contact with a surface, the surface being a sample site (e.g., an assay site, a tissue site, or a combination thereof). In some examples, the surface is at least a portion of a tissue from or within a subject, e.g., in vivo (e.g., living tissue) or ex vivo.

The solvent can, for example, be delivered to the reservoir such that it contacts the sample site non-destructively (e.g., at a force and/or pressure that is non-destructive, such as a low force and/or pressure).

In some examples, the solvent is applied to the sample from the reservoir via a channel independent of the pressurized gas. In some examples, the solvent is applied to the sample under low pressure. For example, the solvent can be applied by a mechanical pump such that solvent is applied to the tissue site (e.g., moved into a reservoir where it is in contact with the tissue site) with minimal force thereby exerting minimal pressure (and producing minimal damage) at a tissue site.

For example, the solvent can be delivered to the reservoir and/or contact the sample site at a pressure of 100 psig or less (e.g., 99 psig or less, 98 psig or less, 97 psig or less, 96 psig or less, 95 psig or less, 90 psig or less, 85 psig or less, 80 psig or less, 75 psig or less, 70 psig or less, 65 psig or less, 60 psig or less, 55 psig or less, 50 psig or less, 45 psig or less, 40 psig or less, 35 psig or less, 30 psig or less, 25 psig or less, 20 psig or less, 15 psig or less, 10 psig or less, 9 psig or less, 8 psig or less, 7 psig or less, 6 psig or less, 5 psig or less, 4.5 psig or less, 4 psig or less, 3.5 psig or less, 3 psig or less, 2.5 psig or less, 2 psig or less, 1.5 psig or less, 1.25 psig or less, 1 psig or less, 0.75 psig or less, 0.5 psig or less, 0.4 psig or less, 0.3 psig or less, 0.2 psig or less, or 0.1 psig or less). In some examples, the solvent can be delivered to the reservoir and/or contact the sample site at a pressure of 0 psig or more (e.g., 0.1 psig or more, 0.2 psig or more, 0.3 psig or more, 0.4 psig or more, 0.5 psig or more, 0.75 psig or more, 1 psig or more, 1.25 psig or more, 1.5 psig or more, 2 psig or more, 2.5 psig or more, 3 psig or more, 3.5 psig or more, 4 psig or more, 4.5 psig or more, 5 psig or more, 6 psig or more, 7 psig or more, 8 psig or more, 9 psig or more, 10 psig or more, 15 psig or more, 20 psig or more, 25 psig or more, 30 psig or more, 35 psig or more, 40 psig or more, 45 psig or more, 50 psig or more, 55 psig or more, 60 psig or more, 65 psig or more, 70 psig or more, 75 psig or more, 80 psig or more, 85 psig or more, 90 psig or more, or 95 psig or more). The pressure at which the solvent is delivered to the reservoir and/or contacted with the sample site can range from any of the minimum values described above to any of the maximum values described above. For example, the solvent can be delivered to the reservoir and/or contact the sample site at a pressure of from 0 psig to 100 psig (e.g., from 0 psig to 50 psig, from 50 psig to 100 psig, from 0 psig to 25 psig, from 25 psig to 50 psig, from 50 psig to 75 psig, from 75 psig to 100 psig, from 0 psig to 20 psig, from 20 psig to 40 psig, from 40 psig to 60 psig, from 60 psig to 80 psig, from 80 psig to 100 psig, from 0 psig to 99 psig, 0 psig to 95 psig, from 0 psig to 90 psig, from 0 psig to 80 psig, from 0 psig to 75 psig, from 0 psig to 60 psig, from 0 psig to 40 psig, from 0 psig to 30 psig, from 0.1 psig to 100 psig, from 5 psig to 100 psig, from 10 psig to 100 psig, from 15 psig to 100 psig, from 20 psig to 100 psig, from 25 psig to 100 psig, from 30 psig to 100 psig, from 40 psig to 100 psig, from 60 psig to 100 psig, from 90 psig to 100 psig, from 0.1 psig to 99 psig, from 5 psig to 95 psig, from 10 psig to 90 psig, from 20 psig to 80 psig, from 30 psig to 70 psig, from 40 psig to 60 psig, from 0.1 psig to 100 psig, from 0.1 psig to 99 psig, 0.1 psig to 95 psig, from 0.1 psig to 90 psig, from 0.1 psig to 80 psig, from 0.1 psig to 75 psig, from 0.1 psig to 60 psig, from 0.1 psig to 40 psig, from 0.1 psig to 30 psig, from 0.1 psig to 25 psig, from 0.1 psig to 20 psig, from 0.1 psig to 15 psig, from 0.1 psig to 10 psig, from 0.1 psig to 5 psig, or from 0.5 psig to 2.5 psig).

In some examples, the apparatus can further comprise a first valve configured to control a flow from the third conduit to the receptacle. The first valve can comprise any suitable type of valve, such as those known in the art. In some examples, the first valve can comprise a pinch valve.

In some examples, the apparatus is configures such that the third conduit is under a vacuum when the first valve is in the open position.

In some examples, the apparatus can further comprise a second valve configured to control a flow of gas through the second conduit. The second valve can comprise any suitable type of valve, such as those known in the art. In some examples, the second valve can comprise a pinch valve.

In some examples, the apparatus can further comprise a pump in fluid communication with the third conduit. The pump can, for example, be configured to increase the velocity of the contents within the third conduit. The pump can comprise any suitable pump, such as those known in the art. For example, the pump can be a peristaltic pump (e.g., a roller pump), a diaphragm pump, a gear pump, a syringe pump, a piston pump, a rotary pump, or a vacuum pump.

In some examples, the apparatus can further comprise a fourth conduit, the fourth conduit being in fluid communication with the receptacle. In some examples, the apparatus can further comprise a pump in fluid communication with the fourth conduit. In some examples, the fourth conduit extends from an inlet to an outlet, the inlet being fluid communication with the receptacle and the outlet being in fluid communication with the pump. The pump can, for example, be configured to increase the velocity of the contents within the third conduit. The pump can comprise any suitable pump, such as those known in the art and those described herein above.

For example, the receptacle is a closed container configured to be fluidly coupled to the third conduit and the fourth conduit. For example, the receptacle can have an interior volume defined by a wall, wherein the wall has one or more ports independently configured to receive the third conduit and the fourth conduit, such that the interior volume of the receptacle is fluidly connected to the third conduit and the fourth conduit. In some examples, the receptacle can comprise a body and a lid, wherein the lid is configured to be coupled to the body (e.g., removably coupled), such that when the lid and the body are coupled they define the interior volume. The lid can, for example, have one or more ports, the one or more ports being configured to receive the third conduit and the fourth conduit.

In some examples, the apparatus can further comprise a waste container in fluid communication with the third conduit. The waste container can comprise any suitable container such as those known in the art and those described herein above. In some examples, the apparatus can further comprise a valve configured to diverge a fluid from the third conduit to the waste container. The valve can comprise any suitable valve, such as those known in the art and those described herein above. In some examples, the apparatus can further comprise a pump configured to remove contents of the waste container. The pump can comprise any suitable pump, such as those known in the art and those described herein above.

In some examples, the probe and/or the apparatus is/are configured to be hand-held (e.g., of an appropriate size and/or shape to be comfortably held in the hand of a user).

In some examples, the apparatus further comprises a housing. The housing can comprise any suitable material, such as a polymer, a resin, a composite material, a ceramic, a metal, or a combination thereof. In some examples, the probe can be disposed within the housing. In some examples, the chamber, the gas supply, the probe, or a combination thereof are disposed within the housing. In some examples, the chamber, the gas supply, and the probe are disposed within the housing.

In some examples, the housing is configured to be hand-held. In some examples, the housing is configured to be disposed within or coupled to a surgical instrument. In some examples, the housing is configured to be disposed within an annulus of a surgical instrument. Examples of surgical instruments include, but are not limited to laparoscopes, trocar needles, biopsy guides, catheters (e.g., multiple-lumen catheters), robotic instruments, and combinations thereof.

In some examples, the apparatus can further comprise a control system configured to be communicatively coupled to one or more of the components disclosed herein. The control system can, for example, comprise a user interface comprising a display showing real-time operating parameters and a control selection panel, and the control selection panel displays control parameters and includes: a selector (e.g., a button, an arrow, a slider, etc.) for starting and stopping an sample collection upon selection by a user; and/or one or more selectors for allowing the user to modify one or more of the control parameters.

For example, the apparatus can further comprise a control system configured to control: a flow of the solvent (e.g., a solvent flow) from the chamber through the first conduit to the reservoir; a flow of the gas (e.g., a gas flow) from the gas supply through the second conduit to the reservoir; a flow of the sample (e.g., a sample flow) from the reservoir through the third conduit to the receptacle; or a combination thereof.

In some examples, the control system is configured to control the solvent flow, such that the solvent flow is at a flow rate of 100 microliters per minute or more (e.g., 125 microliters per minute or more, 150 microliters per minute or more, 175 microliters per minute or more, 200 microliters per minute or more, 225 microliters per minute or more, 250 microliters per minute or more, 275 microliters per minute or more, 300 microliters per minute or more, 325 microliters per minute or more, 350 microliters per minute or more, 375 microliters per minute or more, 400 microliters per minute or more, 425 microliters per minute or more, 450 microliters per minute or more, 475 microliters per minute or more, 500 microliters per minute or more, 550 microliters per minute or more, 600 microliters per minute or more, 650 microliters per minute or more, 700 microliters per minute or more, 750 microliters per minute or more, 800 microliters per minute or more, 850 microliters per minute or more, 900 microliters per minute or more, 950 microliters per minute or more, 1000 microliters per minute or more, 1100 microliters per minute or more, 1200 microliters per minute or more, 1300 microliters per minute or more, 1400 microliters per minute or more, 1500 microliters per minute or more, 1750 microliters per minute or more, 2000 microliters per minute or more, 2250 microliters per minute or more, 2500 microliters per minute or more, 3000 microliters per minute or more, 3500 microliters per minute or more, 4000 microliters per minute or more, or 4500 microliters per minute or more). In some examples, the control system is configured to control the solvent flow such that the solvent flow is at a flow rate of 5000 microliters per minute or less (e.g., 4500 microliters per minute or less, 4000 microliters per minute or less, 3500 microliters per minute or less, 3000 microliters per minute or less, 2500 microliters per minute or less, 2250 microliters per minute or less, 2000 microliters per minute or less, 2000 microliters per minute or less, 1750 microliters per minute or less, 1500 microliters per minute or less, 1400 microliters per minute or less, 1300 microliters per minute or less, 1200 microliters per minute or less, 1100 microliters per minute or less, 1000 microliters per minute or less, 950 microliters per minute or less, 900 microliters per minute or less, 850 microliters per minute or less, 800 microliters per minute or less, 750 microliters per minute or less, 700 microliters per minute or less, 650 microliters per minute or less, 600 microliters per minute or less, 550 microliters per minute or less, 500 microliters per minute or less, 475 microliters per minute or less, 450 microliters per minute or less, 425 microliters per minute or less, 400 microliters per minute or less, 375 microliters per minute or less, 350 microliters per minute or less, 325 microliters per minute or less, 300 microliters per minute or less, 275 microliters per minute or less, 250 microliters per minute or less, 225 microliters per minute or less, 200 microliters per minute or less, 175 microliters per minute or less, 150 microliters per minute or less, or 125 microliters per minute or less). The flow rate of the solvent can range from any of the minimum values described above to any of the maximum values described above. For example, the control system can be configured to control the solvent flow such that the solvent flow is at a flow rate of from 100 to 5000 microliters per minute (e.g., from 100 to 2500 microliters per minute, from 2500 to 5000 microliters per minute, from 100 to 500 microliters per minute, from 500 to 1000 microliters per minute, from 1000 to 5000 microliters per minute, from 100 to 4000 microliters per minute, from 100 to 3000 microliters per minute, from 100 to 2000 microliters per minute, from 100 to 1000 microliters per minute, from 100 to 750 microliters per minute, from 100 to 250 microliters per minute, from 150 to 5000 microliters per minute, from 200 to 5000 microliters per minute, from 250 to 5000 microliters per minute, from 500 to 5000 microliters per minute, from 750 to 5000 microliters per minute, from 2000 to 5000 microliters per minute, from 150 to 4500 microliters per minute, from 150 to 3000 microliters per minute, from 150 to 1000 microliters per minute, from 200 to 800 microliters per minute, or from 200 to 400 microliters per minute).

In some examples, the control system is configured to control the solvent flow, such that the solvent flows for an amount of time of 1 microsecond or more (e.g., 2 microseconds or more, 3 microseconds or more, 4 microseconds or more, 5 microseconds or more, 10 microseconds or more, 15 microseconds or more, 20 microseconds or more, 25 microseconds or more, 30 microseconds or more, 35 microseconds or more, 40 microseconds or more, 45 microseconds or more, 50 microseconds or more, 60 microseconds or more, 70 microseconds or more, 80 microseconds or more, 90 microseconds or more, 100 microseconds or more, 125 microseconds or more, 150 microseconds or more, 175 microseconds or more, 200 microseconds or more, 225 microseconds or more, 250 microseconds or more, 300 microseconds or more, 350 microseconds or more, 400 microseconds or more, 450 microseconds or more, 500 microseconds or more, 600 microseconds or more, 700 microseconds or more, 800 microseconds or more, 900 microseconds or more, 1 millisecond or more, 2 milliseconds or more, 3 milliseconds or more, 4 milliseconds or more, 5 milliseconds or more, 10 milliseconds or more, 15 milliseconds or more, 20 milliseconds or more, 25 milliseconds or more, 30 milliseconds or more, 35 milliseconds or more, 40 milliseconds or more, 45 milliseconds or more, 50 milliseconds or more, 60 milliseconds or more, 70 milliseconds or more, 80 milliseconds or more, 90 milliseconds or more, 100 milliseconds or more, 125 milliseconds or more, 150 milliseconds or more, 175 milliseconds or more, 200 milliseconds or more, 225 milliseconds or more, 250 milliseconds or more, 300 milliseconds or more, 350 milliseconds or more, 400 milliseconds or more, 450 milliseconds or more, 500 milliseconds or more, 600 milliseconds or more, 700 milliseconds or more, 800 milliseconds or more, 900 milliseconds or more, 1 second or more, 2 seconds or more, 3 seconds or more, 4 seconds or more, 5 seconds or more, 10 seconds or more, 15 seconds or more, 20 seconds or more, 25 seconds or more, 30 seconds or more, 35 seconds or more, 40 seconds or more, 45 seconds or more, 50 seconds or more, 55 seconds or more, 1 hour or more, 2 hours or more, 3 hours or more, 4 hours or more, 5 hours or more, 6 hours or more, 7 hours or more, 8 hours or more, 9 hours or more, 10 hours or more, 12 hours or more, 14 hours or more, 16 hours or more, 18 hours or more, 20 hours or more, or 22 hours or more). In some examples, the control system is configured to control the solvent flow, such that the solvent flows for an amount of time of 1 day or less (e.g., 22 hours or less, 20 hours or less, 18 hours or less, 16 hours or less, 14 hours or less, 12 hours or less, 10 hours or less, 9 hours or less, 8 hours or less, 7 hours or less, 6 hours or less, 5 hours or less, 4 hours or less, 3 hours or less, 2 hours or less, 1 hour or less, 55 seconds or less, 50 seconds or less, 45 seconds or less, 40 seconds or less, 35 seconds or less, 30 seconds or less, 25 seconds or less, 20 seconds or less, 15 seconds or less, 10 seconds or less, 5 seconds or less, 4 seconds or less, 3 seconds or less, 2 seconds or less, 1 second or less, 900 milliseconds or less, 800 milliseconds or less, 700 milliseconds or less, 600 milliseconds or less, 500 milliseconds or less, 450 milliseconds or less, 400 milliseconds or less, 350 milliseconds or less, 300 milliseconds or less, 250 milliseconds or less, 225 milliseconds or less, 200 milliseconds or less, 175 milliseconds or less, 150 milliseconds or less, 125 milliseconds or less, 100 milliseconds or less, 90 milliseconds or less, 80 milliseconds or less, 70 milliseconds or less, 60 milliseconds or less, 50 milliseconds or less, 45 milliseconds or less, 40 milliseconds or less, 35 milliseconds or less, 30 milliseconds or less, 25 milliseconds or less, 20 milliseconds or less, 15 milliseconds or less, 10 milliseconds or less, 5 milliseconds or less, 4 milliseconds or less, 3 milliseconds or less, 2 milliseconds or less, 1 millisecond or less, 900 microseconds or less, 800 microseconds or less, 700 microseconds or less, 600 microseconds or less, 500 microseconds or less, 450 microseconds or less, 400 microseconds or less, 350 microseconds or less, 300 microseconds or less, 250 microseconds or less, 225 microseconds or less, 200 microseconds or less, 175 microseconds or less, 150 microseconds or less, 125 microseconds or less, 100 microseconds or less, 90 microseconds or less, 80 microseconds or less, 70 microseconds or less, 60 microseconds or less, 50 microseconds or less, 45 microseconds or less, 40 microseconds or less, 35 microseconds or less, 30 microseconds or less, 25 microseconds or less, 20 microseconds or less, 15 microseconds or less, 10 microseconds or less, or 5 microseconds or less). The amount of time that the solvent flow can range from any of the minimum values described above to any of the maximum values described above. For example, the control system can be configured to control the solvent flow, such that the solvent flows for an amount of time of from 1 microsecond to 1 day (e.g., from 1 microsecond to 1 second, from 1 second to 1 day, from 1 microsecond to 1 millisecond, from 1 millisecond to 1 second, from 1 second to 1 minute, from 1 minute to 1 hour, from 1 hour to 1 day, from 1 microsecond to 18 hours, from 1 microsecond to 12 hours, from 1 microsecond to 6 hours, from 1 microsecond to 1 hour, from 1 microsecond to 30 minutes, from 1 microsecond to 10 minutes, from 1 microsecond to 5 minutes, from 1 microsecond to 1 minute, from 1 microsecond to 30 seconds, from 1 microsecond to 10 seconds, from 1 microsecond to 5 seconds, from 1 microsecond to 1 second, from 1 microsecond to 500 milliseconds, from 1 microsecond to 250 milliseconds, from 1 microsecond to 100 milliseconds, from 1 microsecond to 50 milliseconds, from 1 microsecond to 10 milliseconds, from 1 microsecond to 500 microseconds, from 5 microseconds to 1 day, from 10 microseconds to 1 day, from 25 microseconds to 1 day, from 50 microseconds to 1 day, from 100 microseconds to 1 day, from 500 microseconds to 1 day, from 1 millisecond to 1 day, from 5 milliseconds to 1 day, from 10 milliseconds to 1 day, from 25 milliseconds to 1 day, from 50 milliseconds to 1 day, from 100 milliseconds to 1 day, from 500 milliseconds to 1 day, from 1 second to 1 day, from 5 seconds to 1 day, from 10 seconds to 1 day, from 30 seconds to 1 day, from 1 minute to 1 day, from 5 minutes to 1 day, from 10 minutes to 1 day, from 30 minutes to 1 day, from 1 hour to 1 day, from 2 hours to 1 day, from 4 hours to 1 day, from 6 hours to 1 day, from 12 hours to 1 day, from 5 microseconds to 22 hours, from 10 microseconds to 20 hours, from 50 microseconds to 18 hours, from 100 microseconds to 16 hours, from 500 microseconds to 14 hours, from 1 millisecond to 12 hours, from 5 milliseconds to 10 hours, from 10 milliseconds to 8 hours, from 50 milliseconds to 6 hours, from 100 milliseconds to 4 hours, from 500 milliseconds to 2 hours, from 1 second to 1 hour, from 1 second to 30 minutes, from 1 second to 10 minutes, from 1 second to 1 minute, from 1 second to 30 seconds, from 1 second to 10 seconds, from 1 second to 5 seconds, from 1 second to 3 seconds, from 1 microsecond to 10 seconds, from 1 millisecond to 10 seconds, from 1 microsecond to 5 seconds, or from 1 millisecond to 5 seconds).

In some examples, the control system is configured to control the gas flow, such that the gas flows at a pressure of 0 psig or more (e.g., 0.1 psig or more, 0.2 psig or more, 0.3 psig or more, 0.4 psig or more, 0.5 psig or more, 0.75 psig or more, 1 psig or more, 1.25 psig or more, 1.5 psig or more, 2 psig or more, 2.5 psig or more, 3 psig or more, 3.5 psig or more, 4 psig or more, 4.5 psig or more, 5 psig or more, 6 psig or more, 7 psig or more, 8 psig or more, 9 psig or more, 10 psig or more, 15 psig or more, 20 psig or more, 25 psig or more, 30 psig or more, 35 psig or more, 40 psig or more, 45 psig or more, 50 psig or more, 55 psig or more, 60 psig or more, 65 psig or more, 70 psig or more, 75 psig or more, 80 psig or more, 85 psig or more, 90 psig or more, or 95 psig or more). In some examples, the control system is configured to control the gas flow, such that the gas flows at a pressure of 100 psig or less (e.g., 99 psig or less, 98 psig or less, 97 psig or less, 96 psig or less, 95 psig or less, 90 psig or less, 85 psig or less, 80 psig or less, 75 psig or less, 70 psig or less, 65 psig or less, 60 psig or less, 55 psig or less, 50 psig or less, 45 psig or less, 40 psig or less, 35 psig or less, 30 psig or less, 25 psig or less, 20 psig or less, 15 psig or less, 10 psig or less, 9 psig or less, 8 psig or less, 7 psig or less, 6 psig or less, 5 psig or less, 4.5 psig or less, 4 psig or less, 3.5 psig or less, 3 psig or less, 2.5 psig or less, 2 psig or less, 1.5 psig or less, 1.25 psig or less, 1 psig or less, 0.75 psig or less, 0.5 psig or less, 0.4 psig or less, 0.3 psig or less, 0.2 psig or less, or 0.1 psig or less). The pressure at which the gas flow can range from any of the minimum values described above to any of the maximum values described above. For example, the control system can be configured to control the gas flow, such that the gas flows at a pressure of from 0 psig to 100 psig (e.g., from 0 psig to 50 psig, from 50 psig to 100 psig, from 0 psig to 25 psig, from 25 psig to 50 psig, from 50 psig to 75 psig, from 75 psig to 100 psig, from 0 psig to 20 psig, from 20 psig to 40 psig, from 40 psig to 60 psig, from 60 psig to 80 psig, from 80 psig to 100 psig, from 0 psig to 99 psig, 0 psig to 95 psig, from 0 psig to 90 psig, from 0 psig to 80 psig, from 0 psig to 75 psig, from 0 psig to 60 psig, from 0 psig to 40 psig, from 0 psig to 30 psig, from 0.1 psig to 100 psig, from 5 psig to 100 psig, from 10 psig to 100 psig, from 15 psig to 100 psig, from 20 psig to 100 psig, from 25 psig to 100 psig, from 30 psig to 100 psig, from 40 psig to 100 psig, from 60 psig to 100 psig, from 90 psig to 100 psig, from 0.1 psig to 99 psig, from 5 psig to 95 psig, from 10 psig to 90 psig, from 20 psig to 80 psig, from 30 psig to 70 psig, from 40 psig to 60 psig, from 0.1 psig to 100 psig, from 0.1 psig to 99 psig, 0.1 psig to 95 psig, from 0.1 psig to 90 psig, from 0.1 psig to 80 psig, from 0.1 psig to 75 psig, from 0.1 psig to 60 psig, from 0.1 psig to 40 psig, from 0.1 psig to 30 psig, from 0.1 psig to 25 psig, from 0.1 psig to 20 psig, from 0.1 psig to 15 psig, from 0.1 psig to 10 psig, from 0.1 psig to 5 psig, or from 0.5 psig to 2.5 psig).

In some examples, the control system is configured to control the gas flow, such that the gas flows for an amount of time of 1 microsecond or more (e.g., 2 microseconds or more, 3 microseconds or more, 4 microseconds or more, 5 microseconds or more, 10 microseconds or more, 15 microseconds or more, 20 microseconds or more, 25 microseconds or more, 30 microseconds or more, 35 microseconds or more, 40 microseconds or more, 45 microseconds or more, 50 microseconds or more, 60 microseconds or more, 70 microseconds or more, 80 microseconds or more, 90 microseconds or more, 100 microseconds or more, 125 microseconds or more, 150 microseconds or more, 175 microseconds or more, 200 microseconds or more, 225 microseconds or more, 250 microseconds or more, 300 microseconds or more, 350 microseconds or more, 400 microseconds or more, 450 microseconds or more, 500 microseconds or more, 600 microseconds or more, 700 microseconds or more, 800 microseconds or more, 900 microseconds or more, 1 millisecond or more, 2 milliseconds or more, 3 milliseconds or more, 4 milliseconds or more, 5 milliseconds or more, 10 milliseconds or more, 15 milliseconds or more, 20 milliseconds or more, 25 milliseconds or more, 30 milliseconds or more, 35 milliseconds or more, 40 milliseconds or more, 45 milliseconds or more, 50 milliseconds or more, 60 milliseconds or more, 70 milliseconds or more, 80 milliseconds or more, 90 milliseconds or more, 100 milliseconds or more, 125 milliseconds or more, 150 milliseconds or more, 175 milliseconds or more, 200 milliseconds or more, 225 milliseconds or more, 250 milliseconds or more, 300 milliseconds or more, 350 milliseconds or more, 400 milliseconds or more, 450 milliseconds or more, 500 milliseconds or more, 600 milliseconds or more, 700 milliseconds or more, 800 milliseconds or more, 900 milliseconds or more, 1 second or more, 2 seconds or more, 3 seconds or more, 4 seconds or more, 5 seconds or more, 10 seconds or more, 15 seconds or more, 20 seconds or more, 25 seconds or more, 30 seconds or more, 35 seconds or more, 40 seconds or more, 45 seconds or more, 50 seconds or more, or 55 seconds or more). In some examples, the control system is configured to control the gas flow, such that the gas flows for an amount of time of 1 minute or less (e.g., 55 seconds or less, 50 seconds or less, 45 seconds or less, 40 seconds or less, 35 seconds or less, 30 seconds or less, 25 seconds or less, 20 seconds or less, 15 seconds or less, 10 seconds or less, 5 seconds or less, 4 seconds or less, 3 seconds or less, 2 seconds or less, 1 second or less, 900 milliseconds or less, 800 milliseconds or less, 700 milliseconds or less, 600 milliseconds or less, 500 milliseconds or less, 450 milliseconds or less, 400 milliseconds or less, 350 milliseconds or less, 300 milliseconds or less, 250 milliseconds or less, 225 milliseconds or less, 200 milliseconds or less, 175 milliseconds or less, 150 milliseconds or less, 125 milliseconds or less, 100 milliseconds or less, 90 milliseconds or less, 80 milliseconds or less, 70 milliseconds or less, 60 milliseconds or less, 50 milliseconds or less, 45 milliseconds or less, 40 milliseconds or less, 35 milliseconds or less, 30 milliseconds or less, 25 milliseconds or less, 20 milliseconds or less, 15 milliseconds or less, 10 milliseconds or less, 5 milliseconds or less, 4 milliseconds or less, 3 milliseconds or less, 2 milliseconds or less, 1 millisecond or less, 900 microseconds or less, 800 microseconds or less, 700 microseconds or less, 600 microseconds or less, 500 microseconds or less, 450 microseconds or less, 400 microseconds or less, 350 microseconds or less, 300 microseconds or less, 250 microseconds or less, 225 microseconds or less, 200 microseconds or less, 175 microseconds or less, 150 microseconds or less, 125 microseconds or less, 100 microseconds or less, 90 microseconds or less, 80 microseconds or less, 70 microseconds or less, 60 microseconds or less, 50 microseconds or less, 45 microseconds or less, 40 microseconds or less, 35 microseconds or less, 30 microseconds or less, 25 microseconds or less, 20 microseconds or less, 15 microseconds or less, 10 microseconds or less, or 5 microseconds or less). The amount of time that the gas flows can range from any of the minimum values described above to any of the maximum values described above. For example, the control system can be configured to control the gas flow, such that the gas flows for an amount of time of from 1 microsecond to 1 minute (e.g., from 1 microsecond to 1 millisecond, from 1 millisecond to 1 second, from 1 second to 1 minute, from 1 microsecond to 30 seconds, from 1 microsecond to 10 seconds, from 1 microsecond to 5 seconds, from 1 microsecond to 1 second, from 1 microsecond to 500 milliseconds, from 1 microsecond to 250 milliseconds, from 1 microsecond to 100 milliseconds, from 1 microsecond to 50 milliseconds, from 1 microsecond to 10 milliseconds, from 1 microsecond to 500 microseconds, from 5 microseconds to 1 minute, from 10 microseconds to 1 minute, from 25 microseconds to 1 minute, from 50 microseconds to 1 minute, from 100 microseconds to 1 minute, from 500 microseconds to 1 minute, from 1 millisecond to 1 minute, from 5 milliseconds to 1 minute, from 10 milliseconds to 1 minute, from 25 milliseconds to 1 minute, from 50 milliseconds to 1 minute, from 100 milliseconds to 1 minute, from 500 milliseconds to 1 minute, from 1 second to 1 minute, from 5 seconds to 1 minute, from 10 seconds to 1 minute, from 30 seconds to 1 minute, from 5 microseconds to 45 seconds, from 10 microseconds to 30 seconds, from 50 microseconds to 20 seconds, from 100 microseconds to 15 seconds, from 500 microseconds to 10 seconds, from 1 millisecond to 5 seconds, from 5 milliseconds to 1 second, from 10 milliseconds to 900 milliseconds, from 1 second to 1 minute, from 1 second to 30 seconds, from 1 second to 10 seconds, from 1 second to 5 seconds, from 1 second to 3 seconds, from 1 microsecond to 10 seconds, from 1 millisecond to 10 seconds, from 1 microsecond to 5 seconds, or from 1 millisecond to 5 seconds).

In some examples, the control system is configured to control the sample flow, such that the sample flows for an amount of time of 1 microsecond or more (e.g., 2 microseconds or more, 3 microseconds or more, 4 microseconds or more, 5 microseconds or more, 10 microseconds or more, 15 microseconds or more, 20 microseconds or more, 25 microseconds or more, 30 microseconds or more, 35 microseconds or more, 40 microseconds or more, 45 microseconds or more, 50 microseconds or more, 60 microseconds or more, 70 microseconds or more, 80 microseconds or more, 90 microseconds or more, 100 microseconds or more, 125 microseconds or more, 150 microseconds or more, 175 microseconds or more, 200 microseconds or more, 225 microseconds or more, 250 microseconds or more, 300 microseconds or more, 350 microseconds or more, 400 microseconds or more, 450 microseconds or more, 500 microseconds or more, 600 microseconds or more, 700 microseconds or more, 800 microseconds or more, 900 microseconds or more, 1 millisecond or more, 2 milliseconds or more, 3 milliseconds or more, 4 milliseconds or more, 5 milliseconds or more, 10 milliseconds or more, 15 milliseconds or more, 20 milliseconds or more, 25 milliseconds or more, 30 milliseconds or more, 35 milliseconds or more, 40 milliseconds or more, 45 milliseconds or more, 50 milliseconds or more, 60 milliseconds or more, 70 milliseconds or more, 80 milliseconds or more, 90 milliseconds or more, 100 milliseconds or more, 125 milliseconds or more, 150 milliseconds or more, 175 milliseconds or more, 200 milliseconds or more, 225 milliseconds or more, 250 milliseconds or more, 300 milliseconds or more, 350 milliseconds or more, 400 milliseconds or more, 450 milliseconds or more, 500 milliseconds or more, 600 milliseconds or more, 700 milliseconds or more, 800 milliseconds or more, 900 milliseconds or more, 1 second or more, 2 seconds or more, 3 seconds or more, 4 seconds or more, 5 seconds or more, 10 seconds or more, 15 seconds or more, 20 seconds or more, 25 seconds or more, 30 seconds or more, 35 seconds or more, 40 seconds or more, 45 seconds or more, 50 seconds or more, or 55 seconds or more). In some examples, the control system is configured to control the sample flow, such that the sample flows for an amount of time of 1 minute or less (e.g., 55 seconds or less, 50 seconds or less, 45 seconds or less, 40 seconds or less, 35 seconds or less, 30 seconds or less, 25 seconds or less, 20 seconds or less, 15 seconds or less, 10 seconds or less, 5 seconds or less, 4 seconds or less, 3 seconds or less, 2 seconds or less, 1 second or less, 900 milliseconds or less, 800 milliseconds or less, 700 milliseconds or less, 600 milliseconds or less, 500 milliseconds or less, 450 milliseconds or less, 400 milliseconds or less, 350 milliseconds or less, 300 milliseconds or less, 250 milliseconds or less, 225 milliseconds or less, 200 milliseconds or less, 175 milliseconds or less, 150 milliseconds or less, 125 milliseconds or less, 100 milliseconds or less, 90 milliseconds or less, 80 milliseconds or less, 70 milliseconds or less, 60 milliseconds or less, 50 milliseconds or less, 45 milliseconds or less, 40 milliseconds or less, 35 milliseconds or less, 30 milliseconds or less, 25 milliseconds or less, 20 milliseconds or less, 15 milliseconds or less, 10 milliseconds or less, 5 milliseconds or less, 4 milliseconds or less, 3 milliseconds or less, 2 milliseconds or less, 1 millisecond or less, 900 microseconds or less, 800 microseconds or less, 700 microseconds or less, 600 microseconds or less, 500 microseconds or less, 450 microseconds or less, 400 microseconds or less, 350 microseconds or less, 300 microseconds or less, 250 microseconds or less, 225 microseconds or less, 200 microseconds or less, 175 microseconds or less, 150 microseconds or less, 125 microseconds or less, 100 microseconds or less, 90 microseconds or less, 80 microseconds or less, 70 microseconds or less, 60 microseconds or less, 50 microseconds or less, 45 microseconds or less, 40 microseconds or less, 35 microseconds or less, 30 microseconds or less, 25 microseconds or less, 20 microseconds or less, 15 microseconds or less, 10 microseconds or less, or 5 microseconds or less). The amount of time that the sample flows can range from any of the minimum values described above to any of the maximum values described above. For example, the control system can be configured to control the sample flow, such that the sample flows for an amount of time of from 1 microsecond to 1 minute (e.g., from 1 microsecond to 1 millisecond, from 1 millisecond to 1 second, from 1 second to 1 minute, from 1 microsecond to 30 seconds, from 1 microsecond to 10 seconds, from 1 microsecond to 5 seconds, from 1 microsecond to 1 second, from 1 microsecond to 500 milliseconds, from 1 microsecond to 250 milliseconds, from 1 microsecond to 100 milliseconds, from 1 microsecond to 50 milliseconds, from 1 microsecond to 10 milliseconds, from 1 microsecond to 500 microseconds, from 5 microseconds to 1 minute, from 10 microseconds to 1 minute, from 25 microseconds to 1 minute, from 50 microseconds to 1 minute, from 100 microseconds to 1 minute, from 500 microseconds to 1 minute, from 1 millisecond to 1 minute, from 5 milliseconds to 1 minute, from 10 milliseconds to 1 minute, from 25 milliseconds to 1 minute, from 50 milliseconds to 1 minute, from 100 milliseconds to 1 minute, from 500 milliseconds to 1 minute, from 1 second to 1 minute, from 5 seconds to 1 minute, from 10 seconds to 1 minute, from 30 seconds to 1 minute, from 5 microseconds to 45 seconds, from 10 microseconds to 30 seconds, from 50 microseconds to 20 seconds, from 100 microseconds to 15 seconds, from 500 microseconds to 10 seconds, from 1 millisecond to 5 seconds, from 5 milliseconds to 1 second, from 10 milliseconds to 900 milliseconds, from 1 second to 1 minute, from 1 second to 30 seconds, from 1 second to 10 seconds, from 1 second to 5 seconds, from 1 second to 3 seconds, from 1 microsecond to 10 seconds, from 1 millisecond to 10 seconds, from 1 microsecond to 5 seconds, or from 1 millisecond to 5 seconds).

In some examples, the control system is configured to: control the solvent flow at a flow rate of from 100 and 5000 microliters per minute for a period of time of from 1 microsecond to 1 day; control the gas flow at a pressure of from 0 to 100 psig for a period of time of from 1 microsecond to 1 minute; control the sample flow for a period of time of from 1 microseconds to 1 minute; or a combination thereof.

In some examples, the control system comprises an actuator or haptic control device (e.g., a switch, a pedal, a button, a knob, a lever, a toggle, etc.) that controls solvent flow (e.g., starts and/or stops) upon actuation. For example, in some examples, the control system comprises a trigger or button to initiate solvent flow. In further examples, the control system comprises a pedal (i.e., that can be operated by foot action) to initiate solvent flow.

A skilled artisan will recognize that the lengths of the first and/or second conduit can be adjusted to fit the particular use of the system. In yet further examples, the control system is configured to control: a solvent flow (e.g., flow rate for a fixed period of time) from the chamber through the first conduit to the reservoir.

In some examples, the apparatus can further comprise a cart.

In some examples, the apparatus is not directly coupled to an analyzer (e.g., mass spectrometer).

In some examples, the apparatus is a point-of-care device. In some examples, the apparatus is a handheld apparatus. In some examples, the apparatus is a benchtop apparatus. In some examples, the apparatus is a high-throughput device. For example, the apparatus can be a high-throughput apparatus configured to collect a plurality of liquid samples.

In some examples, the apparatuses detailed herein can be used to collect samples from a wide range of sources. For example, the apparatuses can be used to collect surgical, forensic, agriculture, drug of abuse, pharmaceutical, oil/petroleum samples, or a combination thereof.

In some examples, the materials (PDMS and PTFE) and solvent (e.g., water or sterile water only solvents) used in the devices are biologically compatible, such that they can be used in surgery in for real-time analysis.

Furthermore, because the devices can be very compact, it can be hand-held and used in used in minimally invasive surgical procedures, or non-surgical procedures.

In some examples, the device can be used through cannulas or catheters in minimally invasive surgical or endoscopy procedures, or can be used in non-surgical procedures through needle guides or biopsy guides. In some examples, the devices can integrated to a robotic surgical system, such as the Da Vinci surgical system (e.g., in an automated system). Thus, many regions of the human body cavity can be quickly sampled and analyzed. In some examples, samples collected by the apparatus can be analyzed using a database of molecular signatures and machine learning algorithms, allowing diagnosis in real time for each sampled region. The present invention can be used in a wide variety of oncological and other surgical interventions, such as endometriosis, for which real time characterization and diagnosis of tissues are needed.

In some examples, the probe of the present invention can be used to assist surgeons and medical professionals during minimally invasive surgical interventions by providing comprehensive and definitive diagnostic molecular information in vivo and in real time, without necessarily causing damage or alteration to the patient's native living tissues. For example, the device can demonstrate this capacity during non-laparoscopic/endoscopic surgical procedures.

The apparatuses are also suitable for ex vivo analysis of tissues (fresh, frozen, sections, smears, biopsies) or other clinical specimens that might be examined by a pathologist, and can be used for chemical analysis of any given sample for which direct analysis is desired in confined and spatially limited domains (animals, plants, explosives, drugs, etc.). A variety of tissue types can be analyzed as well, including but not limited to, breast, kidney, lymph node, thyroid, ovary, pancreatic and brain tissues.

In some examples, the probe of the present invention can be used in conjunction with surgical instruments for the treatment of a disease. A variety of surgical instruments can be used to excise or ablate cells or tissues, including, but not limited to, laser ablation tools, tools for cauterization or electrocauterization, or tools for the manual dissection of tissue such as a scalpel.

### Methods of Use

Also disclosed herein are methods of use of any of the apparatuses herein. For example, also disclosed herein are methods for collecting a sample from a surface using any of the apparatuses herein. The methods can, for example, comprise: contacting the probe with the surface; applying a fixed or discrete volume of the solvent to the surface; collecting the applied solvent to obtain a liquid sample; and storing the sample in the receptacle. In some examples, the methods are non-destructive, e.g. to the surface is not damaged. In some examples, the methods can be defined as producing no detectable physical damage to the tissue being assessed.

In some examples, the fixed or discrete volume of solvent is not applied as a spray. In some examples, the fixed or discrete volume of solvent is applied as a droplet (e.g., a single droplet). In a further example, the solvent is applied in a discrete number of droplets from 1 to 10. For example, the solvent can be applied as 1 or more droplets (e.g., 2 or more, 3 or more, 4 or more, 5 or more, 6 or more, 7 or more, 8 or more, or 9 or more). In some examples, the solvent can be applied as 10 of less droplets (e.g., 9 or less, 8 or less, 7 or less, 6 or less, 5 or less, 4 or less, 3 or less, or 2 or less). The number of droplets can range from any of the minimum values described above to any of the maximum values described above. For example, the solvent can be applied in a discrete number of droplets, the number of discrete droplets being from 1 to 10 droplets (e.g., from 1 to 5, from 5 to 10, from 1 to 2, from 2 to 4, from 4 to 6, from 6 to 8, from 8 to 10, from 1 to 9, from 1 to 8, from 1 to 7, from 1 to 6, from 1 to 4, from 1 to 3, from 2 to 10, from 3 to 10, from 4 to 10, from 6 to 10, from 7 to 10, from 8 to 10, from 2 to 9, from 3 to 8, or from 4 to 6). In some examples, the solvent is applied as a single discrete droplet.

The fixed or discrete volume of solvent can, for example, be from 0.01 to 500 μL (e.g., as described above). The fixed or discrete volume of solvent can, for example, be applied using a pressure of 100 psig or less (e.g., 99 psig or less, 98 psig or less, 97 psig or less, 96 psig or less, 95 psig or less, 90 psig or less, 85 psig or less, 80 psig or less, 75 psig or less, 70 psig or less, 65 psig or less, 60 psig or less, 55 psig or less, 50 psig or less, 45 psig or less, 40 psig or less, 35 psig or less, 30 psig or less, 25 psig or less, 20 psig or less, 15 psig or less, 10 psig or less, 9 psig or less, 8 psig or less, 7 psig or less, 6 psig or less, 5 psig or less, 4.5 psig or less, 4 psig or less, 3.5 psig or less, 3 psig or less, 2.5 psig or less, 2 psig or less, 1.5 psig or less, 1.25 psig or less, 1 psig or less, 0.75 psig or less, 0.5 psig or less, 0.4 psig or less, 0.3 psig or less, 0.2 psig or less, or 0.1 psig or less). In some examples, the fixed or discrete volume of solvent is applied using a pressure of 10 psig or less (e.g., 9 psig or less, 8 psig or less, 7 psig or less, 6 psig or less, 5 psig or less, 4.5 psig or less, 4 psig or less, 3.5 psig or less, 3 psig or less, 2.5 psig or less, 2 psig or less, 1.5 psig or less, 1.25 psig or less, 1 psig or less, 0.75 psig or less, 0.5 psig or less, 0.4 psig or less, 0.3 psig or less, 0.2 psig or less, or 0.1 psig or less).

In some examples, collecting the applied solvent comprises applying a negative pressure to pull the sample into the third conduit and/or applying a gas pressure to push the sample into the third conduit and then into the receptacle. In some examples, collecting the applied solvent comprises applying a negative pressure to pull the sample into the third conduit and applying a positive pressure to push the sample into the third conduit and then into the receptacle.

The solvent, for example, is applied through the first conduit that is separate from the third conduit. In some examples, the gas pressure is applied through the second conduit that is separate from the first conduit and the third conduit.

In some examples, applying a gas pressure to push the sample into the third conduit comprises applying a pressure of 100 psig or less (e.g., 99 psig or less, 98 psig or less, 97 psig or less, 96 psig or less, 95 psig or less, 90 psig or less, 85 psig or less, 80 psig or less, 75 psig or less, 70 psig or less, 65 psig or less, 60 psig or less, 55 psig or less, 50 psig or less, 45 psig or less, 40 psig or less, 35 psig or less, 30 psig or less, 25 psig or less, 20 psig or less, 15 psig or less, 10 psig or less, 9 psig or less, 8 psig or less, 7 psig or less, 6 psig or less, 5 psig or less, 4.5 psig or less, 4 psig or less, 3.5 psig or less, 3 psig or less, 2.5 psig or less, 2 psig or less, 1.5 psig or less, 1.25 psig or less, 1 psig or less, 0.75 psig or less, 0.5 psig or less, 0.4 psig or less, 0.3 psig or less, 0.2 psig or less, or 0.1 psig or less).

The surface is a sample site (e.g., an assay site, a tissue site, or a combination thereof). In some examples, the surface is at least a portion of a tissue from or within a subject, e.g., in vivo (e.g., living tissue) or ex vivo, e.g. such that the sample site is a tissue site. In some examples, the method produces no detectable physical damage to the tissue. The methods can, for example, be performed in vivo or ex vivo. In some examples, the tissue site is a portion of the skin of a patient undergoing screening for opioids. In some examples, the tissue site in an internal tissue site that is being surgically assessed. In certain examples, the methods are further defined as an intraoperative method. In some examples, the method does not involve application of ultrasonic or vibrational energy to the tissue. In some examples, the surface and/or the sample site is a portion of a solid object, an inanimate object, or a combination thereof.

In some examples, the solvent is contacted with the surface for an amount of time of 1 microsecond or more before the liquid sample is collected (e.g., 2 microseconds or more, 3 microseconds or more, 4 microseconds or more, 5 microseconds or more, 10 microseconds or more, 15 microseconds or more, 20 microseconds or more, 25 microseconds or more, 30 microseconds or more, 35 microseconds or more, 40 microseconds or more, 45 microseconds or more, 50 microseconds or more, 60 microseconds or more, 70 microseconds or more, 80 microseconds or more, 90 microseconds or more, 100 microseconds or more, 125 microseconds or more, 150 microseconds or more, 175 microseconds or more, 200 microseconds or more, 225 microseconds or more, 250 microseconds or more, 300 microseconds or more, 350 microseconds or more, 400 microseconds or more, 450 microseconds or more, 500 microseconds or more, 600 microseconds or more, 700 microseconds or more, 800 microseconds or more, 900 microseconds or more, 1 millisecond or more, 2 milliseconds or more, 3 milliseconds or more, 4 milliseconds or more, 5 milliseconds or more, 10 milliseconds or more, 15 milliseconds or more, 20 milliseconds or more, 25 milliseconds or more, 30 milliseconds or more, 35 milliseconds or more, 40 milliseconds or more, 45 milliseconds or more, 50 milliseconds or more, 60 milliseconds or more, 70 milliseconds or more, 80 milliseconds or more, 90 milliseconds or more, 100 milliseconds or more, 125 milliseconds or more, 150 milliseconds or more, 175 milliseconds or more, 200 milliseconds or more, 225 milliseconds or more, 250 milliseconds or more, 300 milliseconds or more, 350 milliseconds or more, 400 milliseconds or more, 450 milliseconds or more, 500 milliseconds or more, 600 milliseconds or more, 700 milliseconds or more, 800 milliseconds or more, 900 milliseconds or more, 1 second or more, 2 seconds or more, 3 seconds or more, 4 seconds or more, 5 seconds or more, 10 seconds or more, 15 seconds or more, 20 seconds or more, 25 seconds or more, 30 seconds or more, 35 seconds or more, 40 seconds or more, 45 seconds or more, 50 seconds or more, 55 seconds or more, 1 hour or more, 2 hours or more, 3 hours or more, 4 hours or more, 5 hours or more, 6 hours or more, 7 hours or more, 8 hours or more, 9 hours or more, 10 hours or more, 12 hours or more, 14 hours or more, 16 hours or more, 18 hours or more, 20 hours or more, or 22 hours or more). In some examples, the solvent is contacted with the surface for an amount of time of 1 day or less before the liquid sample is collected (e.g., 22 hours or less, 20 hours or less, 18 hours or less, 16 hours or less, 14 hours or less, 12 hours or less, 10 hours or less, 9 hours or less, 8 hours or less, 7 hours or less, 6 hours or less, 5 hours or less, 4 hours or less, 3 hours or less, 2 hours or less, 1 hour or less, 55 seconds or less, 50 seconds or less, 45 seconds or less, 40 seconds or less, 35 seconds or less, 30 seconds or less, 25 seconds or less, 20 seconds or less, 15 seconds or less, 10 seconds or less, 5 seconds or less, 4 seconds or less, 3 seconds or less, 2 seconds or less, 1 second or less, 900 milliseconds or less, 800 milliseconds or less, 700 milliseconds or less, 600 milliseconds or less, 500 milliseconds or less, 450 milliseconds or less, 400 milliseconds or less, 350 milliseconds or less, 300 milliseconds or less, 250 milliseconds or less, 225 milliseconds or less, 200 milliseconds or less, 175 milliseconds or less, 150 milliseconds or less, 125 milliseconds or less, 100 milliseconds or less, 90 milliseconds or less, 80 milliseconds or less, 70 milliseconds or less, 60 milliseconds or less, 50 milliseconds or less, 45 milliseconds or less, 40 milliseconds or less, 35 milliseconds or less, 30 milliseconds or less, 25 milliseconds or less, 20 milliseconds or less, 15 milliseconds or less, 10 milliseconds or less, 5 milliseconds or less, 4 milliseconds or less, 3 milliseconds or less, 2 milliseconds or less, 1 millisecond or less, 900 microseconds or less, 800 microseconds or less, 700 microseconds or less, 600 microseconds or less, 500 microseconds or less, 450 microseconds or less, 400 microseconds or less, 350 microseconds or less, 300 microseconds or less, 250 microseconds or less, 225 microseconds or less, 200 microseconds or less, 175 microseconds or less, 150 microseconds or less, 125 microseconds or less, 100 microseconds or less, 90 microseconds or less, 80 microseconds or less, 70 microseconds or less, 60 microseconds or less, 50 microseconds or less, 45 microseconds or less, 40 microseconds or less, 35 microseconds or less, 30 microseconds or less, 25 microseconds or less, 20 microseconds or less, 15 microseconds or less, 10 microseconds or less, or 5 microseconds or less). The amount of time that the solvent is contacted with the surface before the liquid sample is collected can range from any of the minimum values described above to any of the maximum values described above. For example, the solvent can be contacted with the surface for an amount of time of from 1 microsecond to 1 day before the liquid sample is collected (e.g., from 1 microsecond to 1 second, from 1 second to 1 day, from 1 microsecond to 1 millisecond, from 1 millisecond to 1 second, from 1 second to 1 minute, from 1 minute to 1 hour, from 1 hour to 1 day, from 1 microsecond to 18 hours, from 1 microsecond to 12 hours, from 1 microsecond to 6 hours, from 1 microsecond to 1 hour, from 1 microsecond to 30 minutes, from 1 microsecond to 10 minutes, from 1 microsecond to 5 minutes, from 1 microsecond to 1 minute, from 1 microsecond to 30 seconds, from 1 microsecond to 10 seconds, from 1 microsecond to 5 seconds, from 1 microsecond to 1 second, from 1 microsecond to 500 milliseconds, from 1 microsecond to 250 milliseconds, from 1 microsecond to 100 milliseconds, from 1 microsecond to 50 milliseconds, from 1 microsecond to 10 milliseconds, from 1 microsecond to 500 microseconds, from 5 microseconds to 1 day, from 10 microseconds to 1 day, from 25 microseconds to 1 day, from 50 microseconds to 1 day, from 100 microseconds to 1 day, from 500 microseconds to 1 day, from 1 millisecond to 1 day, from 5 milliseconds to 1 day, from 10 milliseconds to 1 day, from 25 milliseconds to 1 day, from 50 milliseconds to 1 day, from 100 milliseconds to 1 day, from 500 milliseconds to 1 day, from 1 second to 1 day, from 5 seconds to 1 day, from 10 seconds to 1 day, from 30 seconds to 1 day, from 1 minute to 1 day, from 5 minutes to 1 day, from 10 minutes to 1 day, from 30 minutes to 1 day, from 1 hour to 1 day, from 2 hours to 1 day, from 4 hours to 1 day, from 6 hours to 1 day, from 12 hours to 1 day, from 5 microseconds to 22 hours, from 10 microseconds to 20 hours, from 50 microseconds to 18 hours, from 100 microseconds to 16 hours, from 500 microseconds to 14 hours, from 1 millisecond to 12 hours, from 5 milliseconds to 10 hours, from 10 milliseconds to 8 hours, from 50 milliseconds to 6 hours, from 100 milliseconds to 4 hours, from 500 milliseconds to 2 hours, from 1 second to 1 hour, from 1 second to 30 minutes, from 1 second to 10 minutes, from 1 second to 1 minute, from 1 second to 30 seconds, from 1 second to 10 seconds, from 1 second to 5 seconds, from 1 second to 3 seconds, from 1 microsecond to 10 seconds, from 1 millisecond to 10 seconds, from 1 microsecond to 5 seconds, or from 1 millisecond to 5 seconds).

In some examples, the methods can comprise collecting a plurality of liquid samples. In some examples, the methods can comprise collecting a plurality of liquid samples from a plurality of sites (e.g., 2 or more, 3 or more, 4 or more, 5 or more, 6 or more, 7 or more, 8 or more, 9 or more, or 10 or more sites).

In some examples, the methods can further comprise washing the probe between collection of the different samples. In some examples, the probe is disposable and is changed between collection of the different samples. In some examples, the probe comprises a collection tip and the method further comprises ejecting the collection tip from the probe after the liquid samples are collected.

In some examples, the methods can further comprise replacing the receptacle with an empty receptacle between collection of the different samples.

In some examples, the apparatus can include a plurality of receptacles and the methods can further comprise putting the third conduit in fluid communication with a new receptacle between collection of the different samples.

In some examples, the plurality of tissue sites surrounds a section of tissue that has been surgically resected. In some examples, the resected tissue is a tumor.

In some examples, the methods can further comprise subjecting the sample(s) to analysis to determine a property of the sample(s) and/or the sample site(s). The analysis can, for example, be performed immediately after collection or remotely in location and/or time relative to sample collection.

For example, the methods can comprise removing or decoupling the receptacle (containing the sample) from the apparatus, optionally storing the receptacle containing the sample, and subsequently transporting the receptacle (containing the sample) to an analyzer.

In some examples, the methods can comprise inserting the receptacle containing the sample into an analyzer, such that the receptacle containing the sample is in fluid communication with the analyzer. In some examples, the methods can comprise inserting the apparatus comprising the receptacle containing the sample into an analyzer, such that the receptacle containing the sample is in fluid communication with the analyzer.

The analysis can comprise any suitable analysis performed by any suitable analyzer or instrument. The analysis can, for example, comprise chromatography, spectroscopy, or spectrometry or a combination thereof, such as gas chromatography, liquid chromatography, thin layer chromatography, Raman spectroscopy, UV-vis absorption spectroscopy, IR absorption spectroscopy, fluorescence spectroscopy, mass spectrometry, or a combination thereof.

In some examples, the analysis comprises mass spectrometry. In yet still further examples, the mass spectrometry comprises ambient ionization MS. Mass spectrometry analysis can, for example, comprise determining a profile (e.g., a molecular profile) corresponding to the site. In some examples, the methods can further comprise comparing the profile to a reference profile to determine a property of the sample and/or the sample site.

In some examples, the methods can further comprise subjecting the sample(s) to analysis to determine a property of the sample(s) and/or the sample site(s), wherein the property comprises the presence or absence of an analyte (e.g., one or more analytes) of interest in the sample; the concentration of an analyte (e.g., one or more analytes) of interest in the sample; the identity of the analyte (e.g., one or more analytes) of interest in the sample; or a combination thereof.

In some examples, the analyte of interest is a biomarker (e.g., a molecular indicator associated with a particular pathological or physiological state). In some examples, the biomarker present in the sample can be assayed to identify risk for, diagnosis of, or progression of a pathological or physiological process in a subject. In some examples, the methods can comprise diagnosing and/or monitoring a disease in a subject based on the property of the sample. Examples of diseases include, but are not limited to neurodegenerative diseases, infectious diseases (e.g., infection with a pathogen such as a virus, bacteria, fungi, protozoa, or parasite), rheumatologic diseases, genetic diseases, acute and chronic respiratory diseases, gastrointestinal diseases, liver diseases, dermatologic diseases, cancer, endometriosis, and combinations thereof.

In some examples, the methods can further comprise selecting a course of therapy for the subject based on the property, such as surgery and/or administration of a drug, medicament, or other therapy.

In some examples, the methods can further comprise identifying tissue sites that include diseased tissue. The diseased tissue can, for example, comprise cancer cells. Examples of cancer cells include, but are not limited to, cells or tumor tissues from a thyroid, lymph node, bladder, blood, bone, bone marrow, brain, breast, colon, esophagus, gastrointestine, gum, head, kidney, liver, lung, nasopharynx, neck, ovary, prostate, skin, stomach, testis, tongue, or uterus (or tissues surrounding such tumors). In some examples, the cancer can be a neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; or paragranuloma.

In further examples the cancer is a thyroid cancer, brain cancer (e.g., a glioma), a prostate cancer, a breast cancer (e.g., a triple negative breast cancer), a pancreatic cancer (e.g., a pancreatic ductal adenocarcinoma), acute myeloid leukemia (AML), melanoma, renal cell cancer or a cancer that has metastasized to a lymph node.

In some examples, the diseased tissue can comprise lung, ovarian, thyroid, or breast cancer cells. In some examples, the methods can further comprise resecting tissue sites that are identified to include diseased tissue. In some examples, the methods can further comprise administering an anti-cancer therapy to the subject.

In certain examples, the instant application provides methods and devices for molecular assessment of samples, such as tissue samples. In particular, in some examples, the methods can be used to assess multiple tissue sites during an operation (or biopsy) of the tissue. This feature allows for accurate identification of diseased tissues (e.g., tissue sites retaining cancer cells) in “real-time” allowing surgeons to more accurately address only the diseased tissue relative to surrounding normal tissues. In particular examples, the methods disclosed here can involve delivery of a fixed or discrete volume of solvent to a tissue site, followed by collection of a liquid sample from the site and analysis of the liquid sample by mass spectrometry. Importantly, rather than being applied in a high pressure spray, solvent is applied as discreet droplets and at low pressure. These methods allow for accurate collection of samples from a distinct tissue site while avoiding damage to the tissue being assessed. The resulting mass spectrometry profile from collected samples allows for differentiation of diseased versus normal tissue sites. The method can be repeated at multiple sites of interest to very accurately map molecular changes (e.g., in a tissue). These methodologies can allow assessment of plurality of tissue sites over a short range of time, thereby allowing for very accurate assessment of the boundaries of diseased versus normal tissues.

In some examples, the analyte of interest comprises a chemical, such as a medicament or illegal substance. In some examples, the analyte of interest comprises an opioid (e.g., for a drug screening). Examples of opioids include, but are not limited to, (α/β)-Meprodine; (α/β)-Prodine; 1-(4-Nitrophenylethyl) piperidylidene-2-(4-chlorophenyl) sulfonamide (W-18); 14-Cinnamoyloxycodeinone; 14-Ethoxymetopon; 14-Hydroxydihydrocodeine; 14-Hydroxymorphine; 14-Methoxymetopon; 14-Phenylpropoxymetopon; 18,19-Dehydrobuprenorphine (HS-599); 18-Methoxycoronaridine; 1-Bromocodeine; 1-Chlorocodeine; 1-Iodomorphine Codeine-6-glucuronide; 1-Nitrocodeine; 2,4-Dinitrophenylmorphine; 3-(3-Methoxyphenyl)-3-ethoxycarbonyltropane; 3-(dimethylamino)-2,2-dimethyl-1-phenylpropan-1-one; 3,14-Diacetyloxymorphone; 3,6-Dibutanoylmorphine; 3-Acetyloxymorphone; 3-Allylfentanyl; 3-Hydroxymorphinan; 3-Methylfentanyl; 3-Methylthiofentanyl; 3-Monoacetylmorphine; 4-Chlorophenylpyridomorphinan; 4-Fluoropethidine; 4-Phenylfentanyl; 5,6-Dihydronorsalutaridine; 5,9 alpha-diethyl-2-hydroxybenzomorphan (5,9-DEHB); 6-Acetyldihydromorphine; 6-Keto Nalbuphine; 6-Methyldihydromorphine; 6-Methylenedihydrodesoxymorphine; 6-Monoacetylcodeine; 6-Monoacetylmorphine; 6-Nicotinoyldihydromorphine; 7-Acetoxymitragynine; 7-Hydroxymitragynine; 7-PET; 7-Spiroindanyloxymorphone; 8,14-Dihydroxydihydromorphinone; 8-Carboxamidocyclazocine (8-CAC); Acetorphine; Acetoxyketobemidone; Acetylcodone; Acetyldihydrocodeine; Acetylmethadol; Acetylmorphone; Acetylpropionylmorphine; AD-1211; ADL-5859; AH-7921; Aknadinine; Akuammidine; Akuammine; Alazocine; Alfentanil; Alimadol; Alletorphine (N-allyl-noretorphine); Allylnorpethidine; Allylprodine; Alphaacetylmethadol; Alphamethadol; Alvimopan; Amentoflavone; Anazocine; Anileridine; Anilopam +HCl; Asimadoline; Axomadol; Azaprocin; AZD-2327; Azidomorphine; BDPC; Benzethidine; Benzhydrocodone; Benzylfentanyl; Benzylmorphine; Betacetylmethadol; Betamethadol; Bezitramide; Bisnortilidine; Bremazocine; Brifentanil; BRL-52537; Bromadol; Bromadoline; Bromocodide; Bromoisopropropyldihydromorphinone; Bromomorphide; BU-48; Buprenorphine; Buprenorphine-3-glucuronide; Butinazocine; Butorphanol; Butyrfentanyl; BW373U86; Carbazocine; Carfentanil; Carperidine; Cephakicine; Cephasamine; Chlornaltrexamine; Chlorodihydrocodide; Chloromorphide; Chloroxymorphamine; Ciprefadol; Ciramadol; Clonitazene; Codeine; Codeine methylbromide; Codeine-N-oxide; Codeine-N-oxide (genocodeine); Codeinone; Codide; Codoxime; Cogazocine; Conorfone (codorphone); Coronaridine; Cyclazocine; Cyclorphan; Cyprenorphine; Cyprodime; Cyproterone acetate; Desmethylclozapine; Desmethylmoramide; Desmethylprodine (MPPP); Desocodeine Desomorphine (dihydrodesoxymorphine); Dextromethadone; Dextromoramide; Dextropropoxyphene (propoxyphene); Dezocine; Diacetyldihydromorphine (dihydroheroin, acetylmorphinol); Diampromide; Dibenzoylmorphine; Dibutyrylmorphine; Diethylthiambutene; Difenoxin; Diformylmorphine; Dihydrocodeine; Dihydrocodeine; Dihydrodesoxycodeine (desocodeine); Dihydroetorphine; Dihydroisocodeine; Dihydromorphine; Dimenoxadol; Dimepheptanol (racemethadol); Dimethylmorphine (6-O-Methylcodeine); Dimethylthiambutene; Dioxaphetyl butyrate; Diphenoxylate; Dipipanone; Dipropanoylmorphine; Doxpicomine; DPI-221; DPI-287; DPI-3290; Drotebanol; Droxypropine; Embutramide; Enadoline; Eptazocine; Eseroline; Etazocine; Ethoheptazine; Ethyldihydromorphine; Ethylketazocine; Ethylmethylthiambutene; Ethylmorphine (dionine); Etonitazene; Etorphine; Etoxeridine (carbetidine); Faxeladol; FE 200665; Fedotozine; Fenfangjine G; Fentanyl; Fluorophen; Furethidine; Gemazocine; GR-89696; Herkinorin; Heroin (diacetylmorphine); Heroin-7,8-oxide; Heterocodeine; Hodgkinsine; Homprenorphine; Hydrocodone; Hydromorphinol; Hydromorphone; Hydroxycodeine; Hydroxypethidine (bemidone); HZ-2; Ibazocine; IBNtxA; Ibogaine; IC-26; ICI-199,441; ICI-204,448; Isoaminile; Isocodeine; Isomethadol; Isomethadone; Isotonitazene; Ketamine; Ketazocine; Ketobemidone; Ketorfanol; KNT-42; Kolokol-1; Lefetamine; Levacetylmethadol; Levargorphan; Levoisomethadone; Levomethadone; Levomethorphan; Levomoramide; Levophenacylmorphan; Levopropoxyphene; Levorphanol; Lofentanil; Loperamide; LPK-26; LS-115509; Lufuradom; Matrine; MCOPPB; Menthol; Meperidine-N-oxide; Meptazinol; Metazocine; Metethoheptazine; Methadone; Metheptazine; Methorphan (racemethorphan); Methyldesorphine; Methyldihydromorphine (dihydroheterocodeine); Methyldihydromorphinone; Methylketobemidone; Metofoline; Metonitazene; Metopon; Mirfentanil; Mitragynine; Mitragynine pseudoindoxyl; Morphanol (racemorphanol); Morphenol; Morpheridine; Morphine; Morphine methylbromide; Morphine-6-glucuronide; Morphine-N-oxide; Morphine-N-oxide (genomorphine); Morphinone; Morphol; Moxazocine; MT-45; MT-7716; Myrophine; Nalbuphine; Nalbuphone; Nalfurafine; Nalorphine; Nalorphine dinicotinate; Naltrexol; N-cyclopropylmethylnoretorphine; Nepenthone; Nexeridine; Nicocodeine; Nicodicodeine; Nicomorphine; N-Methylcarfentanil; N-Methylmorphinan; NNC 63-0532; Noracymethadol; Norbuprenorphine; Norbuprenorphine-3-glucuronide; Norcodeine; Noribogaine; Norlevorphanol; Normethadone; Normorphine; Noroxymorphone; Norpipanone; Norpropoxyphene; Nortilidine; N-Phenethyl-14-ethoxymetopon; N-Phenethyl-14-ethoxymetopon; N-Phenethylnordesomorphine; N-Phenethylnormorphine; Ocfentanil; O-Desmethyltramadol; Ohmefentanyl; Opium; Oripavine; Oxilorphan; Oxpheneridine (carbamethidine); Oxycodone; Oxymorphazone; Oxymorphol; Oxymorphone; Pantopon; Papaveretum (Omnopon); Parafluorofentanyl; Pentamorphone; Pentazocine; PEPAP; Pericine; Pethidine (meperidine); Phenadone; Phenadoxone (heptazone); Phenampromide; Phenaridine; Phenazocine; Phencyclidine; Pheneridine; Phenomorphan; Phenoperidine; Pholcodine (morpholinylethylmorphine); Picenadol; Piminodine; Piperidylthiambutene; Piritramide; Prodilidine; Profadol; Proglumide; Proheptazine; Properidine (ipropethidine); Propiram; Propylketobemidone; Prosidol; Proxorphan; Pseudoakuammigine; Pseudomorphine; Pyrrolidinylthiambutene; Pyrroliphene; PZM21; Quadazocine; R-30490; R-4066; Racemoramide; RAM-378; Remifentanil; Ro-1539; Ro4-1539; Ro64-6198; Ro65-6570; RWJ-394,674; Salvinorin A; Salvinorin B ethoxymethyl ether; Salvinorin B methoxymethyl ether; Sameridine; SB-612,111; SC-17599; Semorphone; SKF-10047; SNC-80; SoRI-9409; Spiradoline; SR-16435; SR-8993; Sufentanil; TAN-67; Tannagine; Tapentadol; Tetrapon; Thebacon; Thebacon (acetyldihydrocodeinone, dihydrocodeinone enol acetate); Thebaine; Thenylfentanyl; Thevinone; Thiambutene; Thiazocine; Thienorphine; Thiobromadol (C-8813); Thiofentanyl; Tifluadom; Tilidine; Tonazocine; Tramadol; Transisocodeine; Trefentanil; Trimebutine; Trimeperidine (promedol); U-47700; U-50,488; U-69,593; Viminol; Volazocine; Zenazocine; α-Chlorocodide; α-Chloromorphide; a-hydrocodol; α-Methylacetylfentanyl; α-Methylfentanyl; α-Methylthiofentanyl; β-Chlorocodide; β-hydroxyfentanyl; β-hydroxythiofentanyl; β-Methylfentanyl; ψ-Akuammigine; derivatives thereof; and combinations thereof.

In some examples, the methods detailed herein can be used to collect and analyze samples from a wide range of sources. For example, the methods can be used to assess surgical, forensic, agriculture, drug of abuse, pharmaceutical, oil/petroleum samples, or a combination thereof.

In some examples, the present disclosure provides methods of determining the presence of diseased tissue (e.g., tumor tissue) or detecting a molecular signature of a biological specimen by identifying specific patterns of a mass spectrometry profile. Biological specimens for analysis can be from animals, plants, or any material (living or non-living) that has been in contact with biological molecules or organisms. A biological specimen can be samples in vivo (e.g., during surgery) or ex vivo.

A profile obtained by the methods can correspond to, for example, proteins, metabolites, or lipids from analyzed biological specimens or tissue sites. These patterns can be determined by measuring the presence of specific molecules using mass spectrometry. Some non-limiting examples of ionizations methods that can be used for the analysis of the sample(s) include chemical ionization, laser ionization, atmospheric-pressure chemical ionization, electron ionization, fast atom bombardment, electrospray ionization, thermal ionization. Additional ionization methods include inductively coupled plasma sources, photoionization, glow discharge, field desorption, thermospray, desorption/ionization on silicon, direct analysis in real time, secondary ion mass spectroscopy, spark ionization, and thermal ionization.

In particular, the methods can further comprise using an ambient ionization source or method for obtaining the mass spectral data such as extraction ambient ionization source. Extraction ambient ionization sources are methods with, in this case, liquid extraction processes dynamically followed by ionization. Some non-limiting examples of extraction ambient ionization sources include air flow-assisted desorption electrospray ionization (AFADESI), direct analysis in real time (DART), desorption electrospray ionization (DESI), desorption ionization by charge exchange (DICE), electrode-assisted desorption electrospray ionization (EADESI), electrospray laser desorption ionization (ELDI), electrostatic spray ionization (ESTASI), Jet desorption electrospray ionization (JeDI), laser assisted desorption electrospray ionization (LADESI), laser desorption electrospray ionization (LDESI), matrix-assisted laser desorption electrospray ionization (MALDESI), nanospray desorption electrospray ionization (nano-DESI), or transmission mode desorption electrospray ionization (TM-DESI).

As with many mass spectrometry methods, ionization efficiency can be optimized by modifying the collection or solvent conditions such as the solvent components, the pH, the gas flow rates, the applied voltage, capillary temperature, and other examples which affect ionization of the sample solution. In particular, the present methods contemplate the use of a solvent or solution which is compatible with human tissue. Some non-limiting examples of solvent which can be used as the ionization solvent include water, ethanol, methanol, acetonitrile, dimethylformamide, an acid, or a mixture thereof. In some examples, the method contemplates a mixture of acetonitrile and dimethylformamide. The amounts of acetonitrile and dimethylformamide can be varied to enhance the extraction of the analytes from the sample as well as increase the ionization and volatility of the sample. In some examples, the composition contains from about 5:1 (v/v) dimethylformamide: acetonitrile to about 1:5 (v/v) dimethylformamide:acetonitrile such as 1:1 (v/v) dimethylformamide: acetonitrile. However, in certain examples the solvent for use is a pharmaceutically acceptable solvent, such as sterile water or a buffered aqueous solution.

### Example Devices

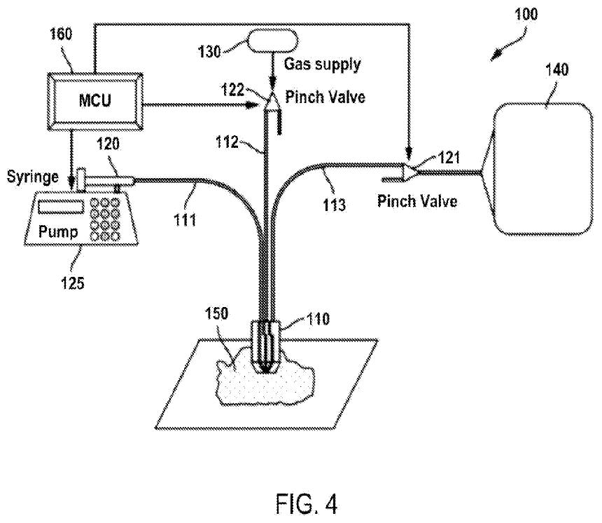

Referring initially to FIG. 4, an apparatus 100 is shown for producing a sample for analysis. In this example, apparatus 100 comprises a probe 110, a chamber 120 with solvent, a gas supply 130 and a receptacle 140. In some examples, the probe is comprised in housing (e.g., to provide a grip in the case of a hand-held device). In further examples, the housing can comprise clicker feature (e.g., a trigger, button, or pedal) that can be used to control fluid and/or gas flow through the probe. In some examples, the probe is composed of a material comprising PDMS, PTFE, SLA 3D printed elastomer, or a combination thereof. In some examples, the probe is produced by a 3D printing process.

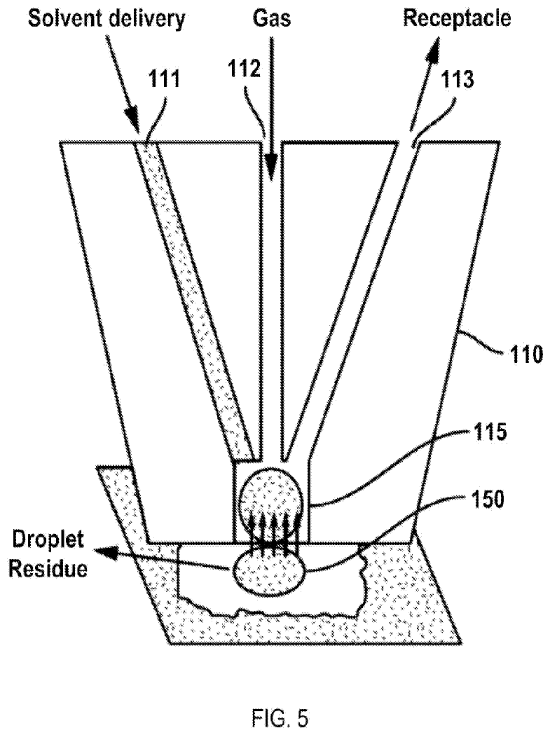

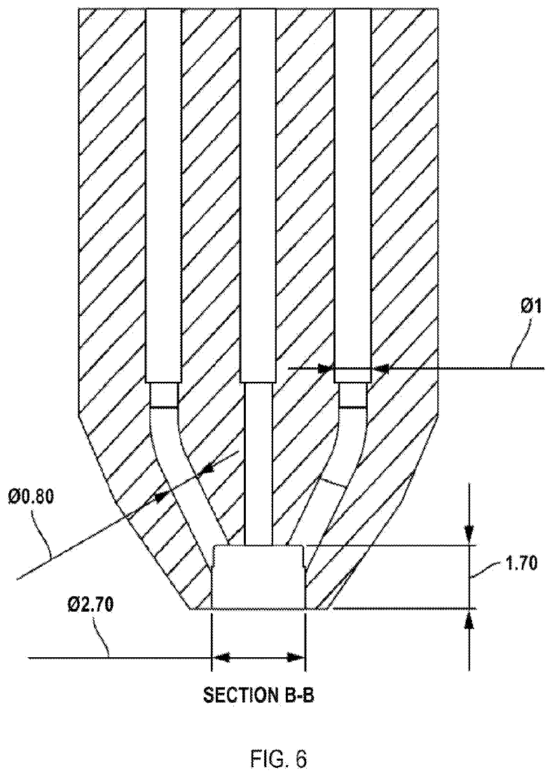

FIG. 5 provides a more detailed cross-section view of probe 110 and illustrates probe 110 comprises a first conduit 111, a second conduit 112, a third conduit 113 and a reservoir 115. In the illustrated example, first conduit 111 is in fluid communication with chamber 120, second conduit 112 is in fluid communication with gas supply 130, and third conduit 113 is in fluid communication with the receptacle 140. FIG. 6 provides an additional cross-section view of a probe with dimensions for a particular example.

It is understood that in certain examples, each of conduits 111, 112, and 113 (which can be of any desired length) can comprise separate components. For example, the portion of each of the conduits within probe 110 can be formed as integral channels during the manufacturing of probe 110. In addition, the portions of each of the conduits between probe 110 and chamber 120, gas supply 130, and receptacle 140 can be tubing or other components suitable for providing fluid flow.

In this example, apparatus 100 can comprise a pump 125 configured to transfer the solvent from chamber 120 to the first conduit 111 and reservoir 115. In the example shown, apparatus 100 can also comprise a first valve 121 configured to control a sample flow from reservoir 115 through third conduit 113 to the receptacle 140. Apparatus 100 can also comprise a second valve 122 configured to control a flow of gas through second conduit 112 to reservoir 115.

A control system 160 can be configured to control operating parameters of apparatus 100. For example, control system 160 can be configured to control a flow of solvent from chamber 120 through first conduit 111 to reservoir 115 by controlling the operation of pump 125. In addition, control system 160 can be configured to control the sample flow from reservoir 115 to receptacle 140 by controlling the opening and closing of first valve 121. Control system 160 can further be configured to control the gas flow from gas supply 130 to reservoir 115 by controlling the opening and closing of second valve 122.

During operation of apparatus 100, a user can position probe 110 so that reservoir 115 is placed on sample site 150. Control system 160 can operate pump 125 for specific periods of time to transfer a desired volume of the solvent from chamber 120 to reservoir 115 via first conduit 111. In some examples, the solvent in chamber 120 can assist in the efficient extraction of molecules from the sample site 150 (e.g., a tissue sample site) for analysis.

In addition, control system 160 can allow a particular period of time between the operation of pump 125 and the opening of first valve 121. This can allow a vacuum (e.g., a separate vacuum system) to draw sample materials (e.g., molecules from tissue sample site 150) from reservoir 115 to the receptacle 140 via third conduit 113.

When first valve 121 is opened, control system 160 can also open second valve 122 to allow a gas (e.g., air, argon, N₂, and/or CO₂) to be transferred from the gas supply 130 to reservoir 115 via second conduit 112. The gas can assist in sample tissue drying prior to analysis, as well as prevent a solvent gap in first conduit 111 (e.g., as a result of a vacuum pulled when reservoir 115 contacts sample site 150). The gas can also assist in solvent transport from sample site 150 to receptacle 140 through third conduit 113.

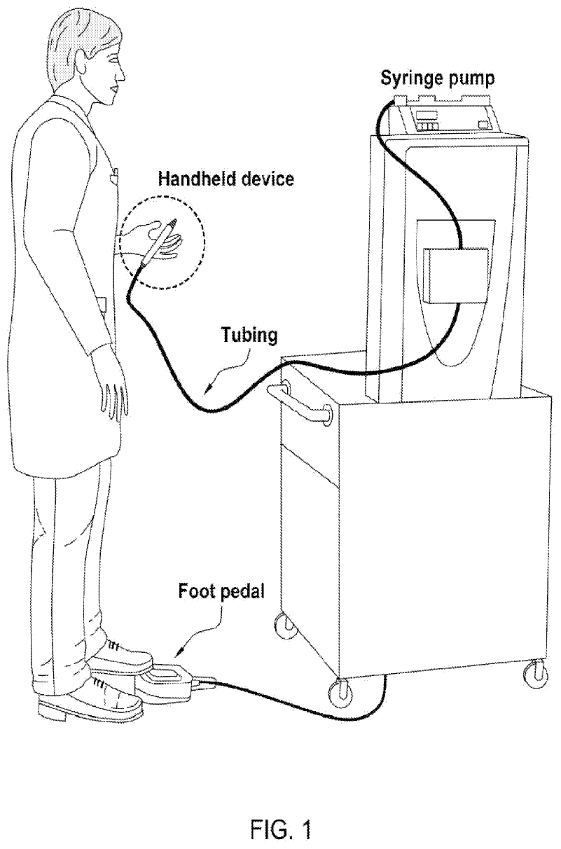

Control system 160 can comprise software and hardware suitable for operating the various components of apparatus 100. Particular examples of the various components shown in the schematic of FIG. 1-FIG. 8 are provided in the examples discussed below, including the section entitled Example 1.

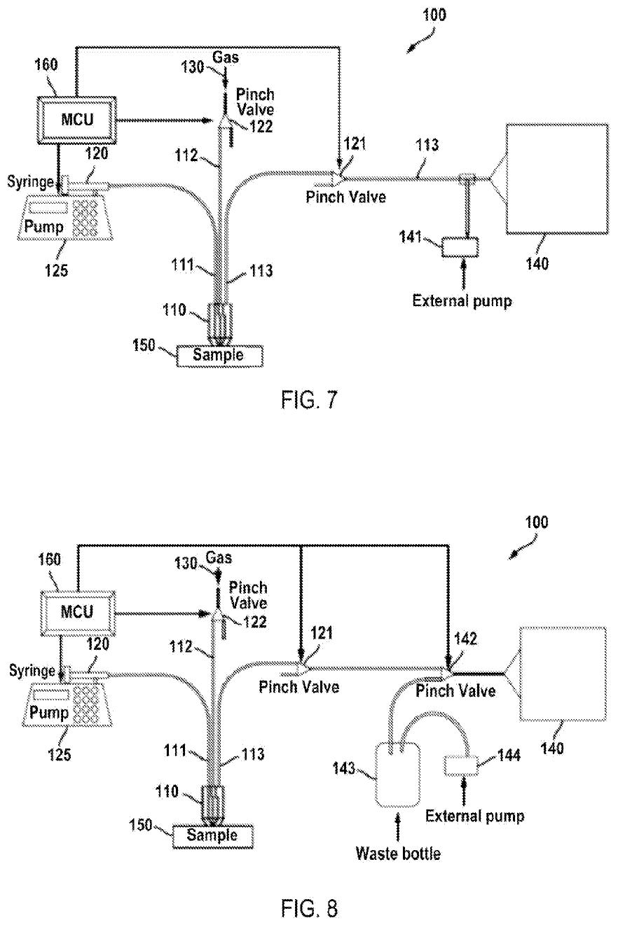

FIG. 7 illustrates an example of apparatus 100 that is similar to the example shown in the previous FIG. 4. In the example of FIG. 7, however, apparatus 100 further comprises a pump 141 in fluid communication with conduit 113. In certain examples, pump 141 can be an external vacuum pump that can be operated to increase the velocity of the sample portion through conduit 113 to the receptacle. It is understood that the components of apparatus 100 described in previous examples operate in an equivalent manner in this example (and subsequently described examples). For purposes of clarity, not all components are labeled with reference numbers in each of the figures. In addition, the operational examples of components that are equivalent to components in previously-described examples will not be repeated in the discussion of this or subsequent examples.

FIG. 8 illustrates another example of apparatus 100 that is similar to the previously-described examples but also comprises a valve 142, a waste container 143 and a pump 144 in fluid communication with conduit 113. In certain examples, valve 142 can be used to diverge a solvent or other cleaning solution from conduit 113 to waste container 143 during cleaning steps. Waste container 143 can be emptied via operation of pump 144. In some examples, cleaning or washing steps using water, ethanol, mixtures of water and ethanol at any ratio, as well as other solvents can be used at any stage of sample analysis to decrease carry over effects.

In certain examples, probe 110 can also be switched between each use. Further, probe 110 can be inserted into a vial containing solvent for washing step using gas (bubbling) to assist with cleaning before or after the automatic wash step. Other cleaning methods including wiping with a sterile solution can also be used. For example, the methods can use a cleaning protocol of: 1. Replace probe; 2. Wash with solution of 50/50 ethanol/water; 3. Wash with 100% ethanol.

FIG. 34 illustrates another example of apparatus 100 that is similar to the previously described examples but also comprises a flow constrictor coupled to at least a portion of the first conduit. The flow constrictor can be operated to deliver precise solvent volumes. Use of the flow constrictor can, for example, affect the liquid sample. For example, using the flow constrictor can affect the presence and/or concentration of lipid(s) within the liquid sample. It is understood that the components of apparatus 100 described in previous examples operate in an equivalent manner in this example (and subsequently described examples). For purposes of clarity, not all components are labeled with reference numbers in each of the figures. In addition, the operational examples of components that are equivalent to components in previously-described examples will not be repeated in the discussion of this or subsequent examples.

### Computing Device

Any of the methods disclosed herein can be carried out in whole or in part on one or more computing or processing devices.

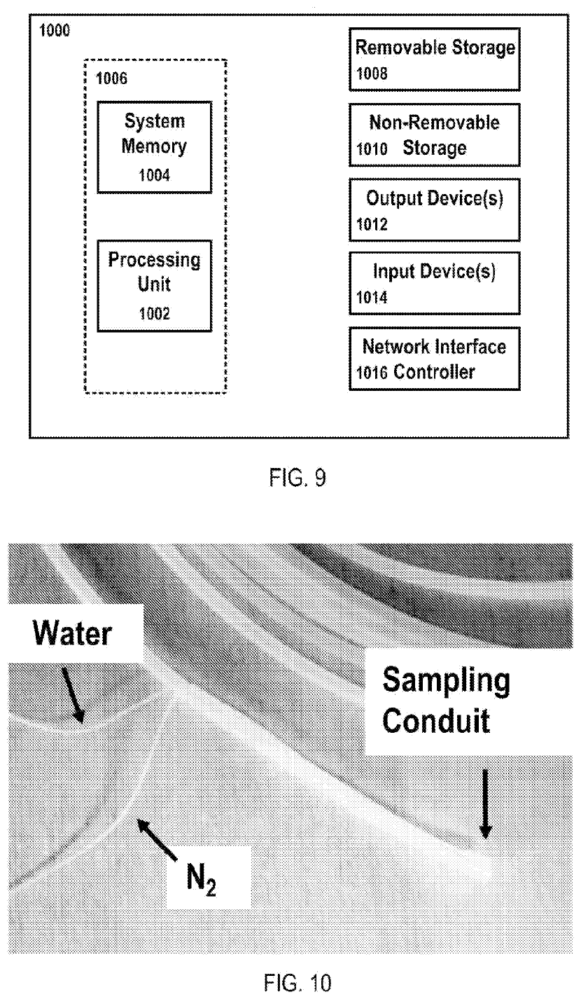

FIG. 9 illustrates an example computing device 1000 upon which examples disclosed herein may be implemented. The computing device 1000 can include a bus or other communication mechanism for communicating information among various components of the computing device 1000. In its most basic configuration, computing device 1000 typically includes at least one processing unit 1002 (a processor) and system memory 1004. Depending on the exact configuration and type of computing device, system memory 1004 may be volatile (such as random access memory (RAM)), non-volatile (such as read-only memory (ROM), flash memory, etc.), or some combination of the two. This most basic configuration is illustrated in FIG. 9 by a dashed line 1006. The processing unit 1002 may be a standard programmable processor that performs arithmetic and logic operations necessary for operation of the computing device 1000.

The computing device 1000 can have additional features/functionality. For example, computing device 1000 may include additional storage such as removable storage 1008 and non- removable storage 1010 including, but not limited to, magnetic or optical disks or tapes. The computing device 1000 can also contain network connection(s) 1016 that allow the device to communicate with other devices. The computing device 1000 can also have input device(s) 1014 such as a keyboard, mouse, touch screen, antenna or other systems configured to communicate with the camera in the system described above, etc. Output device(s) 1012 such as a display, speakers, printer, etc. may also be included. The additional devices can be connected to the bus in order to facilitate communication of data among the components of the computing device 1000.

The processing unit 1002 can be configured to execute program code encoded in tangible, computer-readable media. Computer-readable media refers to any media that is capable of providing data that causes the computing device 1000 (i.e., a machine) to operate in a particular fashion. Various computer-readable media can be utilized to provide instructions to the processing unit 1002 for execution. Common forms of computer-readable media include, for example, magnetic media, optical media, physical media, memory chips or cartridges, a carrier wave, or any other medium from which a computer can read. Example computer-readable media can include, but is not limited to, volatile media, non-volatile media, and transmission media. Volatile and non-volatile media can be implemented in any method or technology for storage of information such as computer readable instructions, data structures, program modules or other data and common forms are discussed in detail below. Transmission media can include coaxial cables, copper wires and/or fiber optic cables, as well as acoustic or light waves, such as those generated during radio-wave and infra-red data communication. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 1002 can execute program code stored in the system memory 1004. For example, the bus can carry data to the system memory 1004, from which the processing unit 1002 receives and executes instructions. The data received by the system memory 1004 can optionally be stored on the removable storage 1008 or the non-removable storage 1010 before or after execution by the processing unit 1002.

The computing device 1000 typically includes a variety of computer-readable media. Computer-readable media can be any available media that can be accessed by device 1000 and includes both volatile and non-volatile media, removable and non-removable media. Computer storage media include volatile and non-volatile, and removable and non-removable media implemented in any method or technology for storage of information such as computer readable instructions, data structures, program modules or other data. System memory 1004, removable storage 1008, and non-removable storage 1010 are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing device 1000. Any such computer storage media can be part of computing device 1000.

It should be understood that the various techniques described herein can be implemented in connection with hardware or software or, where appropriate, with a combination thereof. Thus, the methods, systems, and associated signal processing of the presently disclosed subject matter, or certain aspects or portions thereof, can take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs can implement or utilize the processes described in connection with the presently disclosed subject matter, e.g., through the use of an application programming interface (API), reusable controls, or the like. Such programs can be implemented in a high level procedural or object-oriented programming language to communicate with a computer system. However, the program(s) can be implemented in assembly or machine language, if desired. In any case, the language can be a compiled or interpreted language and it may be combined with hardware implementations.

In certain examples, the methods can be carried out in whole or in part on a computing device 1000 comprising a processor 1002 and a memory 1004 operably coupled to the processor 1002, the memory 1004 having further computer-executable instructions stored thereon that, when executed by the processor 1002, cause the processor 1002 to carry out one or more of the method steps described above.

A number of embodiments of the invention have been described. Nevertheless, it will be understood that various modifications may be made without departing from the spirit and scope of the invention. Accordingly, other embodiments are within the scope of the following claims.

The examples below are intended to further illustrate certain aspects of the systems and methods described herein, and are not intended to limit the scope of the claims.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of measurement conditions, e.g., component concentrations, temperatures, pressures and other measurement ranges and conditions that can be used to optimize the described process.

### Example 1

The MasSpec Pen (FIG. 1) was developed as an automated and biocompatible handheld sampling probe that allows gentle, time-and volume-controlled extraction of molecules from a tissue sample using a discrete water droplet. Several prototypes of the system were engineered with the goal of minimizing tissue damage, maximizing tissue-analyte extraction, and maximizing solvent transfer to the receptacle.

The system developed comprises three main parts: 1) a syringe pump that is programmed to deliver a discrete solvent volume using a controlled flow rate; 2) tubing systems integrated to two-way pinch valves for controlled solvent transport; 3) a probe tip which is used for direct sampling of biological tissues. Several iterations of the system were explored and optimized with the ultimate goal of minimizing tissue damage, maximizing tissue-analyte extraction, and maximizing solvent transmission to the receptacle. FIG. 1 shows a schematic figure of one example of an apparatus comprising a Diagnostic Pen (MasSpec Pen) device for analyzing biological tissue.

The optimized system contains three primary components: 1) a syringe pump that is programmed to deliver a defined water volume (4-10 μL) to the sampling probe; 2) small diameter (ID 800 μm) polytetrafluoroethylene (PTFE) tubing conduits which are integrated to a fast (8 ms) two-way pinch valves for controlled solvent transport from pump to tissue, and from the tissue to the mass spectrometer; 3) a handheld pen-sized probe for direct sampling of biological tissues.

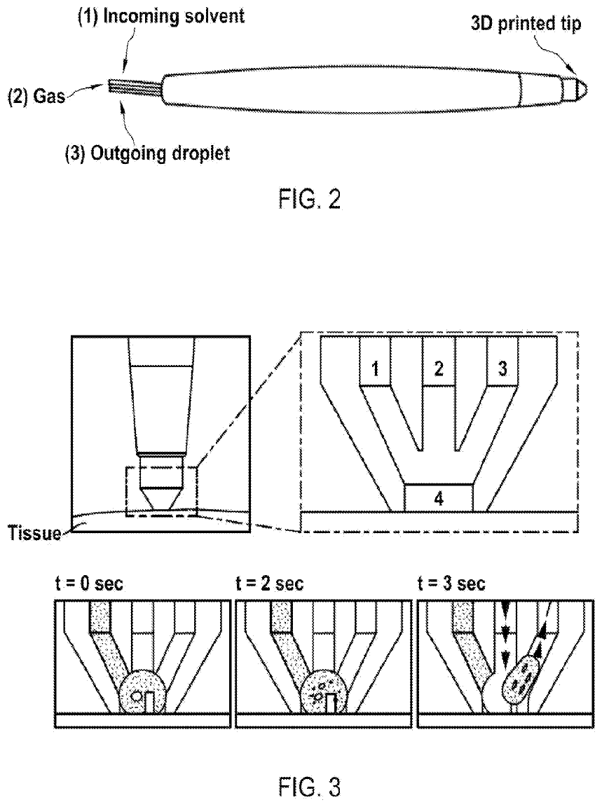

The main component of the handheld pen-sized probe is a 3D-printed polydimethylsiloxane (PDMS) tip (FIG. 2) in which the solvent is retained during interaction with the tissue. The tip was manufactured using 3D-printing and is made of biologically compatible polydimethylsiloxane (PDMS). The tip is designed with three main ports: one for the incoming (solvent) conduit system (tube 111 or conduit 1), a central port for gas (e.g., argon, N₂, CO₂, air) delivery (tube 112 or conduit 2), and an outgoing port to transport molecular constituents in the water droplet from tissue to the receptacle (tube 113 or conduit 3). At the probe tip, all ports combine into a small reservoir where the single droplet is retained and exposed to the tissue sample for a controlled amount of time (e.g., 3 s), allowing efficient analyte extraction. The diameter of the reservoir determines the volume of solvent exposed to the tissue as well as the spatial resolution of the device. Using current tooling, MasSpec Pen tips were designed with sampling sizes ranging from 1.5 mm to 5.0 mm, which is determined by the reservoir diameter. At a 2.77 mm reservoir diameter, a solvent volume of 10 μL is retained in the reservoir and contacted to the tissue sample for a defined time period, while 4.4 μL are retained in a reservoir with a 1.5 mm diameter. After the 3 s extraction period, the MasSpec Pen is removed from the tissue. Concomitantly, conduit 3 is opened allowing vacuum extraction of the droplet to the receptacle, a positive pressure from a low-pressure gas delivery (<10 psi) is provided through conduit 2, followed by a flush step to clean the system. Note that contact times of 1 second, 3 seconds and 5 seconds between the droplet and the tissue sample were evaluated. The 3 seconds contact time was selected for all the experiments as it allowed ease of operation by the user and yielded mass spectra with sufficient total ion intensity. The gas provided by the second tube does not participate in the extraction process, but is used instead to prevent collapse of the system due to the vacuum employed and to assist solvent transport from tissue to the receptacle. Similarly, the flush step is not used for extraction of biomolecules from tissues as there is no contact with tissue during this period.

The three conduit tubes used are made of polytetrafluoroethylene (PTFE), which is also biologically compatible. Tube 111 is used to deliver solvent from syringe pump to the probe tip. Tube 112 is used, in some cases, to deliver a gas (e.g., air, argon, N₂, or CO₂) to the probe tip. The gas serves three main purposes: 1) tissue drying prior to analysis; 2) prevent solvent gap in tube 111 due to the mass spectrometer's vacuum when the reservoir is closed by contacting the tissue specimen; 2) assist solvent transport from tissue to the mass spectrometer through tube 113. However, in some circumstances there is no need for use of a gas. Tube 113 is connected to the receptacle, and a positive pressure of a system can be s used to drive the droplet from the reservoir to the receptacle.

The time events involved in the device operation are automated and precisely controlled by software that communicates with an Arduino system and two two-way pinch valves. All pinch valves are closed until the process is initiated when: 1. under 300 μL/min, a pulse is sent to the pump to infuse the solvent for two seconds and stop, generating a 10 μL droplet filling in the MasSpec Pen reservoir; 2. Tubes 112 and 113 are closed, allowing the solvent in the reservoir to interact with the tissue for three seconds to extract the molecules; 3. The pinch valves controlling tubes 112 and 113 are opened simultaneously, allowing the droplet to transfer to the receptacle for collection. 4. A pulse is sent to the pump to infuse the solvent for another 12 seconds and stop, to completely drive all the extracted molecules into the receptacle. 5. Leave tube 112 and 113 open for another 20 seconds to allow all the solvent in tube 113 to go into the receptacle. The total time is 37 seconds.

The tip design using three conduit tubes and high speed actuated pinch valves allowed precise control of droplet motion and showed excellent performance and robustness. The entire process can be completed in 10 s or less and can be fully automated using an Arduino microcontroller, so that each acquisition is individually triggered through a one-step click using a foot pedal. System automation ensures that each solvent droplet is delivered separately to the receptacle. After each use, the MasSpec Pen can be cleaned if residues are observed through a rapid and automated cleaning flush, or by replacing the disposable tip.

Several solvent systems can be used in the device. In this example, to assure full biological compatibility of the device, water was used as the only solvent, although mixtures of ethanol and water in different ratios were also explored and yielded similar results.

The MasSpec Pen was designed to operate directly on tissue specimens independently of tissue stiffness and morphology. For example, the MasSpec Pen device can operate on fresh tissue samples independently of morphology.

The performance of the MasSpec Pen was tested to analyze soft tissue samples (0.1-5 g) from different organs. The MasSpec Pen tip was gently contacted to the surface of the tissue sample for a period of 3 s while extraction took place. The extraction process at the tissue surface efficiently occurs independently on the tissue shape and rigidity.

Visual and microscopic inspection of all the tissue samples after MasSpec Pen analysis revealed no detectable damage to the tissue sample morphology in the region probed. No observable damage to the tissue was seen at the region analyzed. Note that the automated and time-controlled operational steps of the MasSpec Pen prevents tissue damage as the tissue is only exposed to the small water droplet and not to the vacuum used to transport the droplet from the reservoir to the receptacle. Thus, the MasSpec Pen is a non-destructive approach to obtain rich molecular information from tissue samples.

Because all the materials (e.g., PDMS and PTFE) and solvent (e.g., only water) used in the MasSpec Pen design are biologically compatible, the system has a high potential to be used in surgery in handheld way for real-time analysis. More than that, due to the small dimension of the device, it can even be integrated to a robotic surgical system, such as the Da Vinci surgical system through an accessory port or one of its robotic arms. Several regions of the human body cavity can be quickly sampled during surgery, and analyzed by using a database of molecular signatures and machine learning algorithms. Therefore, the diagnosing results can be provided in real time for each sampled region. This system can be broadly used in a wide variety of oncological and other surgical interventions (such as endometriosis) for which real-time characterization and diagnosis of tissues are needed.

Design and Engineering of the MasSpec Pen: A 3D printer (Model uPrint SE plus) was used to print the key component—PDMS (Dow Corning, Midland, MI, USA) probe tip. The pen tips were fabricated by casting an elastomer from a negative mold and then dissolving the mold away. The negative molds were designed using SolidWorks computer aided design (CAD) software and then fused deposition modeled with the 3D printer using ABS plastic (Stratasys, Eden Prairie, MN, USA) and soluble support material. The parts were then washed to remove support material, using a support cleaning apparatus (SCA-1200HT, SCA) and solvent (EcoWorks) at 70° C. for 24 hrs or until support material was fully dissolved. For the casting, a mixture of PDMS elastomer base and curing agent (Sylgard 184, Dow Corning) were prepared in a weight ratio of 10:1, respectively. The mixture was poured into the 3-D printed molds, cured in an oven (10GCE-LT, Quincy Lab) at 74° C. for 1 h, and then placed in a closed container with acetone (Fisher Scientific, Waltham, MA, USA) to dissolve. The final washing step had the tips sonicated in acetone to remove any remaining ABS. PTFE tubing (ID 1/32inch, OD 1/16inch, Cole-Parmer, Vernon Hills, IL, USA) was directly inserted into the probe tip for experiments.

### Example 2

In some examples, the tubing systems and probe tip can be integrated into a minimally invasive surgical device such as a cannula or catheter for use in laparoscopic or endoscopic surgeries. Several iterations of the system were explored and optimized with the ultimate goal of minimizing tissue damage, maximizing tissue-analyte extraction, and maximizing solvent transmission to the receptacle.

FIG. 4 shows a schematic figure of one example of an apparatus for collecting a sample from a tissue. The syringe pump feeds solvent and gas into the probe via tubing, such as micro-PTFE tubing or medical grade tubing. The probe maintains contact with the sample, retains solvent during interaction with the tissue. The tip was manufactured using 3D-printing and is made of biologically compatible polydimethylsiloxane (PDMS). The probe has three main ports: one for the incoming tubing system, a central port for gas delivery, and a third for the outgoing tubing system. All ports come in junction at a small reservoir where the droplet is retained and exposed to the tissue sample for a controlled amount of time, allowing for efficient extraction of molecules. The size of the reservoir determines the spatial resolution of the device. A solvent volume of 10 μL is exposed to the tissue sample. FIG. 10 shows the three conduit tubes. The three conduit tubes used are made of polytetrafluoroethylene (PTFE), which is also biologically compatible. The tube from the syringe pump is used to deliver solvent from syringe pump to the probe tip, while the other micro-PTFE tube is used to deliver a gas (e.g., air, argon, N₂, or CO₂) to the probe tip. The gas serves three main purposes: 1) tissue drying prior to analysis; 2) prevent solvent gap due to vacuum when the reservoir is closed by contacting the tissue specimen; 2) assist solvent transport from tissue to the receptacle through the wider PTFE tubing. The larger PTFE tubing is directly connected to the receptacle so that a positive pressure from a vacuum system can be used to drive the droplet from the reservoir to the receptacle.

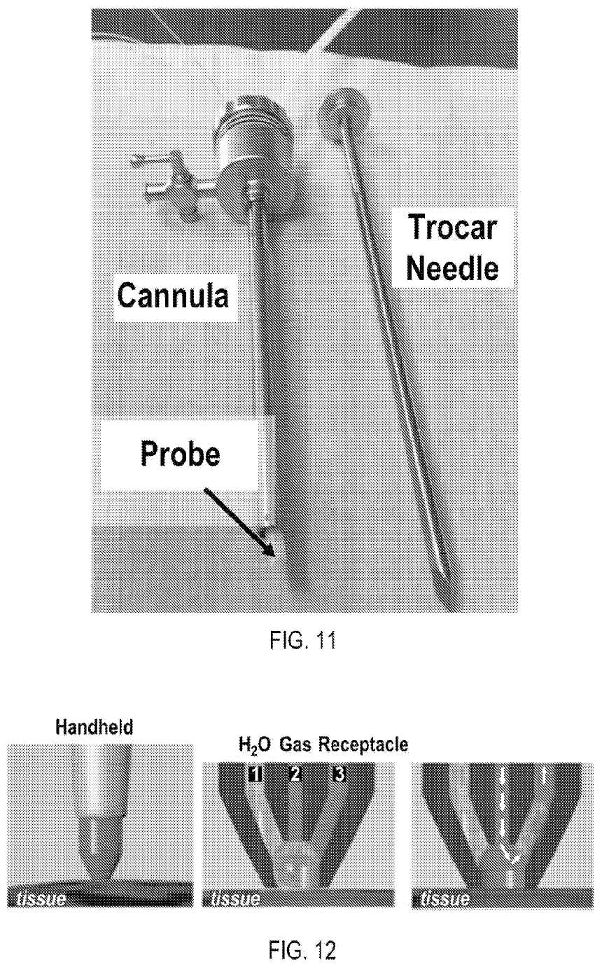

FIG. 11 shows two of the possible devices to house the probe. The cannula shown has the gas and solvent tubing entering the top, as well as the tubing to the receptacle. The probe is shown emerging from the bottom of the cannula. The probe can also be introduced into the body cavity using a trocar needle.

The time events involved in the device operation are automated and precisely controlled by software that communicates with an Arduino system and two two-way pinch valves. All pinch valves are closed until the process is initiated when, under 300 μL/min, a pulse is sent to the pump to infuse the solvent for two seconds and stop, generating a 10 μL droplet filling in the probe reservoir. The gas and collection tubes are closed at pinch valves, allowing the solvent in the reservoir to interact with the tissue for three seconds to extract the molecules. The pinch valves controlling the gas and collection tubes are opened simultaneously, allowing the droplet to transfer to the receptacle for collection. A pulse is sent to the pump to infuse the solvent for another 12 seconds and stop, to completely drive all the extracted molecules into the receptacle. The gas and collection tubes are left open for another 20 seconds to allow all the solvent in the collection tube to go into the receptacle. The total time is 37 seconds.

The probe can be washed between analyses in a variety of methods. Generally, the tip of the probe is wiped with sterile water.

Because of the nature of minimally invasive surgical techniques, the diameter of tubing, and length of tubing can be of particular importance.

The handheld MasSpec Pen has a diameter of 10 mm, which was dictated by the diameter of the 3D printed polydimethylsiloxane (PDMS) pen tip. The tip of the handheld MasSpec Pen was designed with three conduits (incoming water, incoming gas, and outgoing water), which are in fluid communication with an open reservoir that positions the water droplet for contact with tissue surface (FIG. 12).

### Example 3—Portable/Disconnected MasSpec Pen as Sampling Device

The MasSpec Pen was previously developed as a cancer detection device for intrasurgical, in vivo, and/or non-destructive real time diagnosis of tissues (U.S. Pat. No. 10,643,832). The MasSpec Pen uses small volume of liquid solvent delivered to pen tip to extract analytes from sample surface. The solution containing extracted analytes is rapidly pulled by mass spectrometer vacuum for chemical analysis.

Herein, a version of the MasSpec Pen system is described wherein extracted analyte solution is collected within the MasSpec Pen for immediate or later analysis with added advantages of portability, better reproducibility, and sensitivity. For example, a version of the MasSpec Pen is described that is used as a portable/collection device for sampling, disconnected from the final measurement method, i.e. the mass spectrometry. In this method, the sample solution on MasSpec Pen tip is collected in a receptacle within the MasSpec Pen rather than transported directly to the mass spectrometer. The sample solution can be immediately analyzed, or analyzed at a later time point with mass spectrometry or other analytical methods, depending on the application.

Collecting analyte solution within the MasSpec Pen rather than sending it directly to a mass spectrometer provides many advantages. The sample solution can be subjected to different analytical methods such as ESI-MS, paper-spray-MS, HPLC-MS, etc. The collected sample can be introduced to a mass spectrometer with more efficient ionization sources, such as ESI, which can lead to higher sensitivity. To increase the throughput of the analysis with ESI, injection of the sample via a six-port valve or autosampler can be used, which will only add very small amount of time for the analysis. Further, since the MasSpec Pen is used separately for sample collection, the mass spectrometer does not need to be in the same room, which is advantageous for clinical and intrasurgical applications or many other uses where space/resources might be limited. Eliminating the long tubing connected to the mass spectrometer with shorter tubing connected to small external vacuum pump reduces the irreproducibility caused by problems with droplet transfer process. In addition, for applications in which immediate analysis is not necessary or not possible, collected sample solution can be stored and shipped to a facility of a lab where the mass spectrometer is for later analysis.

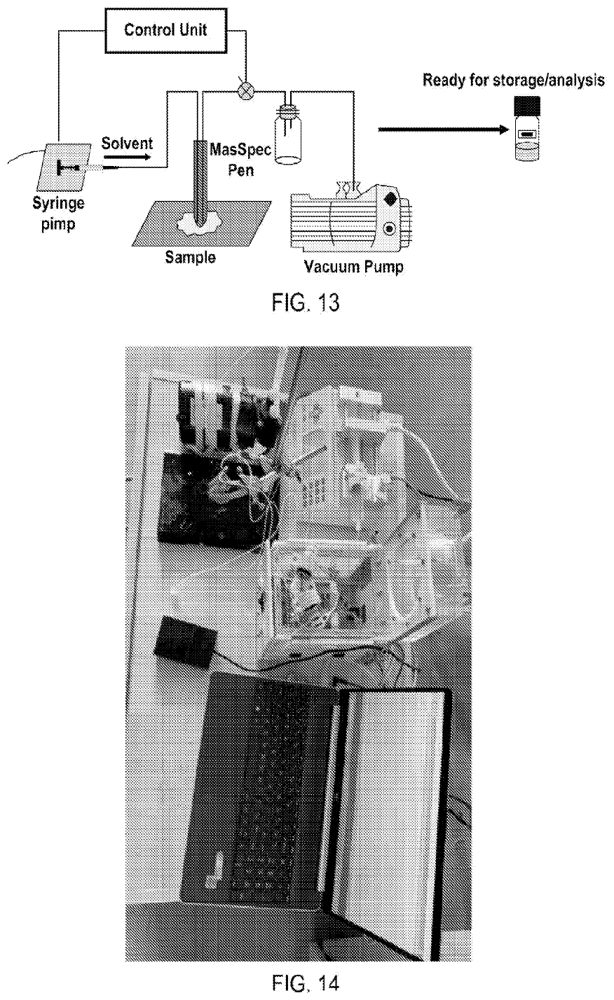

A compact design in which small external vacuum pump and MasSpec Pen are assembled are envisioned to be used as a portable system for sample collection (FIG. 13). The system has been evaluated with six-port valve injection system for ESI-MS for samples such as mouse brain and ovarian cancer tissues which shows high throughput analysis (one analysis within less than one minute) with very high reproducibility (˜5% RSD). A 5 fg/mL cardiolipin standard was detected with signal-to-noise ratio of 36 with six-port valve injection for ESI-MS. Furthermore, the system provides the flexibility to add suitable reagents to the sample solution before mass spectral analysis to improve the sensitivity. For example: addition of base (sodium hydroxide) to tissue extract with MasSpec Pen showed improved ion signal for lipids (e.g., m/z 885.55, PI 38:4).

The MasSpec Pen device is a simple and effective technique to extract analyte molecules from a sample surface. Analysis with mass spectrometer adds the advantages of high sensitivity and selectivity. Specifically, the tissue analysis is performed in a laboratory environment and involves complex sample preparation and separation method. This disconnected MasSpec Pen is portable and easy to use for tissue analysis. This disconnected MasSpec Pen design provides flexibility of immediate or later mass spectral analysis. The extracted analyte solution can also be divided and subjected to different analytical methods, for example, ESI-MS for qualitative analysis and HPLC-MS for quantitative analysis. Since the sample solution can be transferred to anywhere, the analyte solution can be subjected to higher resolution mass spectrometers for more accurate results. The handling of the disconnected MasSpec Pen device is simple, which allows even non-professionals to use the disconnected MasSpec Pen to collect sample solution. This portable and easy to use disconnected MasSpec Pen design expands the utility of the technique to a greater number of applications, such as those where results are not time sensitive, along with an advantage of better sensitivity.

The original MasSpec Pen needs the mass spectrometer directly connected to the MasSpec Pen, which adds some challenges, especially in rooms with limited space/resources and in remote places where mass spectrometers are not available. The disconnected MasSpec Pen can be used in the presence or absence of a mass spectrometer, depending on the application. For an operating room (OR) scenario, the mass spectrometer can be placed in a nearby room so that analysis can be done immediately after sample collection, e.g., within minutes. For remote places, samples can be collected, stored, and shipped for later analysis in a lab. Furthermore, better sensitivity can be achieved by introducing the collected sample solution to more efficient ionization sources. Sensitivity can be further improved by adding suitable reagents to the collected sample before mass spectral analysis. This can particularly be important when a small number of analytes are extracted to the solvent, for example, in the case of in vivo skin analysis. Therefore, this allows for the MasSpec Pen to be used in a variety of applications while still maintaining the simplicity of the system, including employment in the fieldable applications.

Tissue analysis can be very challenging and time consuming. Tumor margin evaluation methods takes long time (>30 minutes) and requires trained pathologists. Even then there are large errors in pathologic evaluation. Devices like MasSpec pen can provide these results in real time with the help of highly specific mass spectral data and machine learning techniques. The disconnected MasSpec Pen allows for the sample solution to be collected within the MasSpec Pen with the freedom of subsequently using any suitable analytical technique. For example, a six-port valve injection ESI-MS needs only 5 μL of sample solution, which means the rest of the sample solution can be used for other analysis. Analyzing the sample solution in both positive- and negative-ion mode mass spectrometry allows for better characterization of chemical content of the sample. Another advantage of the disconnected MasSpec Pen is that it does not need to be directly connected to the mass spectrometer, which gives the freedom for portable applications. Depending on the application, the analysis of collected sample can be done immediately after collection if desired. Since the mass spectrometer does not physically need to be close to the MasSpec Pen, the mass spectrometer does not need to be dedicated for only MasSpec Pen analysis, which eventually makes the analysis cheaper.

Other mass spectrometry (MS) based methods that can also be portable are paper spray MS, direct analysis in real time (DART)-MS, and wooden tip MS. All these techniques are alignment dependent and need significant control of the ionization sources to get reproducible data. In addition, DART-MS is a plasma-based technique which makes it unsuitable for tissue analysis (e.g., because it is destructive). The MasSpec Pen, however, is handheld, biocompatible, easy to use, and can allow for sample analysis in any geometry. After sampling, the analyte solution can be introduced to the detector via an ESI source that is permanently positioned in the correct orientation. Use of a six-port valve can increase the throughput of the analysis (<1 minute per analysis). For forensic applications, for example, immediate analysis can be performed with portable a mass spectrometer for screening purposes. Then the remaining portion of sample solution can again be used for more accurate and/or quantitative analysis in the laboratory setting if needed.

The process of analyte extraction in the MasSpec Pen is liquid-solid extraction, which might not be equally efficient for all analytes. However, this can be overcome by using different solvents whenever suitable. The device can be used in many applications other than tissue analysis. For example, this device can be used for chemical analysis of any given sample (animals, plants, pharmaceutical products, etc.).

### Example 4—Portable MasSpec Pen as Sampling Tool

Mass spectrometers are costly and can take up a lot of space and the vacuum pumps from mass spectrometers can produce sounds that can be disturbing in many environments, such as in an operating room (OR). In addition, there are very few techniques that can be coupled with mass spectrometry for rapid tissue analysis. In a similar way, there are very few techniques that can analyze samples in remote places accurately with high sensitivity and selectivity. This is because powerful instruments, like high resolution-mass spectrometers, can be bulky and it is not always feasible to transport them to remote locations. In both cases, a sampling device is needed which can extract and collect the analytes from a sample surface, wherein said analytes can then be analyzed with a mass spectrometer available in the immediate vicinity, in a nearby vicinity (e.g., next door), or at a location far from the sampling location, depending on the application.

A MasSpec Pen device was previously developed which allows a solvent droplet to extract analytes from a sample surface and then directly sends the sample to a mass spectrometer for chemical analysis. Herein, a design is described wherein the MasSpec Pen is disconnected from mass spectrometer and the analyte solution can be collected in a receptacle within the MasSpec Pen. An external vacuum pump can draw analyte solution from the MasSpec Pen tip to a receptacle. The collected solution is then introduced to a mass spectrometer using any ionization method. Herein, electrospray ionization via a six-port valve injection was used. The disconnected MasSpec Pen provides freedom to place a mass spectrometer close to the MasSpec Pen device or far from it. It also allows for the sample to be introduced through more efficient ionization sources, like ESI. Analyte solution can be introduced into the mass spectrometer via a six-port valve to achieve high throughput. If better sensitivity is required, suitable reagents can be added to the sample to improve the ion signal. In addition, analyte solutions can be analyzed with both positive- and negative-ion mode mass spectrometry for better characterization of the sample. Shorter tubing needed to collect the analyte solution, definite volume introduction via six-port valve, and efficient ionization by ESI provides together can also provide more reproducible results. Additionally, the reproducibility of the signal obtained allows for relative quantification of these analytes.

Described herein is a MasSpec pen device in which the pen is disconnected from the mass spectrometer and allows sampling and storage of the analyte solution for immediate analysis or analysis in a later time and/or separate location.

The disconnected MasSpec Pen allows analysis of samples in remote locations, allows for the use of mass spectral methods with higher sensitivity and specificity, and provides freedom to have mass spectrometer (or other analytical technique) onsite or in a remote location. The possibility of using the MasSpec Pen for just sample collection also makes it less expensive, as it does not require that the mass spectrometer be dedicated only to one MasSpec Pen device.

### Example 5—Detecting Drugs of Abuse: Development and Application of the Modular MasSpec Pen for Opioid Screening of Patient Samples

Described herein is the development and application of the modular MasSpec Pen system for rapid, direct, and sensitive opioid screening of patient skin samples.

Introduction. The opioid epidemic is an alarming societal crisis. The global prevalence of opioid-use has dramatically increased by a reported 76% in the last decade and opioid overdose deaths have nearly doubled in the USA. Yet, advances in sensitive, reliable mass spectrometry methods for opioid screening are limited, with many screenings requiring time-consuming sample preparation and struggling with the evolving opioid analog landscape. Screening can thus take days to complete and, with growing demands, the delays are steadily increasing. Therefore, there is pressing need for methods that allow direct and rapid point-of- care sampling and analysis for opioid detection. Herein, the development of a modular MasSpec Pen system, incorporating a disconnected sampling and analysis platform, for opioid screening is described.

More specifically, a method using the modular MasSpec Pen system for opioid screening on skin was developed and evaluated. the performance of the method was evaluated on a few common opioids and LODs for these opioids were estimated using pure standards. A proof-of-concept study to evaluate the opioid screening capability of the method on human skin was performed.

Methods. Standards were purchased from Cerilliant (Round Rock, TX). For each analyte, eight calibrants were prepared in 1:1 methanol:water by serial dilution. Five 5 μL droplets of each calibrant were deposited on PTFE-coated glass slides. Using the modular MasSpec Pen system and a 4 mm diameter MasSpec Pen tip, dried calibrants were sampled and collected into glass vials. The collected droplets were then analyzed using six-port valve ESI on a Thermo QExactive HF Orbitrap. Human skin samples were obtained from the Cooperative Human Tissue Network and used in a proof-of-concept study to evaluate the system for opioid screening. Lastly, the system was used for opioid screening at Johns Hopkins Medical School on patient skin samples under an approved IRB protocol.

Results. MasSpec Pen analysis involves delivery of a solvent droplet to the pen tip where the solvent desorbs molecules from a sample surface, and the droplet is then directly transported to the mass spectrometer for analysis. To expand the applications of the technology, a modular version of the MasSpec Pen was developed in which the droplets, instead of being directly analyzed, are transported to a collection vial and stored for later analysis (FIG. 13-FIG. 14).

The process of analyte extraction in the MasSpec Pen is liquid-solid extraction, which might not be equally efficient for all analytes. However, this can be overcome by using different solvents whenever suitable. Accordingly, a solvent optimization study was performed using the modular MasSpec Pen. The variables tested included the composition of the extraction solvent, the composition of the ionization solvent, and the scan mode used for analysis. Extraction solvents tested were MeOH:H₂O (control), H₂O, EtOH:H₂O 1:1, EtOH:H₂O 20:80, and EtOH:H₂O 5:95. The ionization solvents tested were MeOH:H₂O with 0.1% acetic acid, and 100% MeOH with 0.1% Acetic acid. Both full scan and MSMS modes were used for comparison.

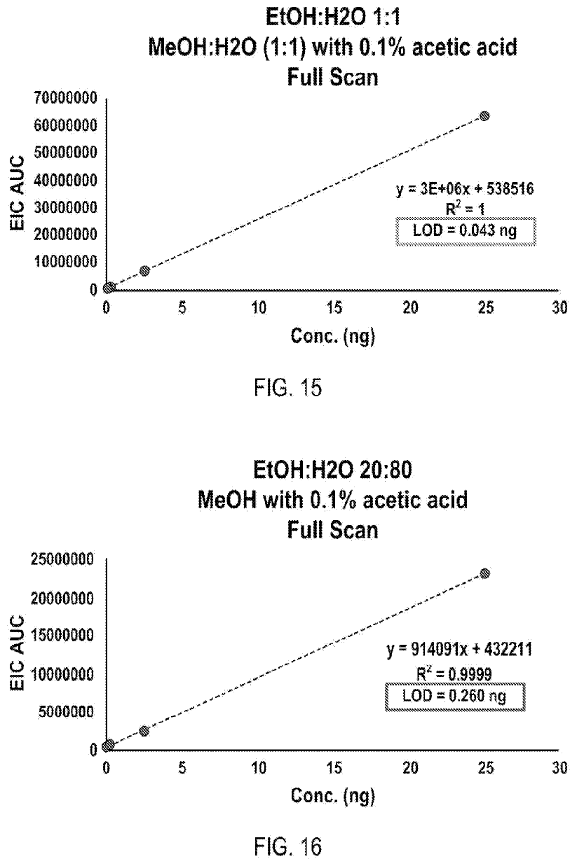

For the method, truncated calibration curves were made (0.025 ng, 0.25 ng, 2.5 ng, and 25 ng) and the performance was compared (FIG. 15-FIG. 16). The results indicated that the best combination of the variables based on performance were an extraction solvent of ethanol:water 1:1, an ionization solvent of methanol:water (1:1) with 0.1% acetic acid, and full scan mode, which provided a limited of detection of 0.043 ng (FIG. 15). The second best combination of variables based on performance were an extraction solvent of ethanol:water 20:80, an ionization solvent of methanol with 0.1% acetic acid, and full scan mode, which results in a limit of detection of 0.26 ng (FIG. 16).

Next, the reproducibility of the analytical performance of the modular MasSpec Pen was evaluated. To assess the reproducibility of the collected sample volume, 9 vials were collected using the modular MSPen system (3 droplets per vial) and the total volume was calculated. The results were that the average collected volume per vial was 73±2 μL. The sample stability was assessed by collecting 5 ppm of a pure oxycodone standard into 5 vials (3 droplets per vial) and the samples were injected and analyzed on day 1, 10, 20, and 30 after storage in −20° C.

RSD of analysis (analysis reproducibility) was assessed by collecting 5 ppm of a pure oxycodone standard into 5 vials (3 droplets per vial), then injecting and analyzing each vial 4 times, and finding the % RSD of each vial. The results were that the average % RSD for multiple analyses from the same vial was 0.17% ±0.09%.

The RSD of sample from a human sample (complex matrix sampling reproducibility) was also assessed. Droplets were collected from human skin tissue into 15 vials. Each vial was then injected and analyzed. The % RSD was then calculated using all of the vials. The result was that the average % RSD for multiple samples collected from the same sample was 9.4%.

The RSD of sampling from a pure standard (pure small molecule sample reproducibility) was also assessed. 5 ppm of a pure oxycodone standard was collected into 9 vials (3 droplets per vial). Each vial was then injected and analyzed. The % RSD was then calculated using all of the vials. The result was that the average % RSD for multiple samples collected from the same sample was 3.2%±2.1%.

The analytical performance of the modular MasSpec Pen was evaluated using autopsy skin samples and good sampling reproducibility (RSD of 9.4%, n=15) and sample stability (cosine similarity values of 0.85 to 0.97 for samples analyzed at 1 and 29 days) was achieved. Additionally, an RSD of 3.1% ±2.1% (n=18) was calculated using the area under the curve for two concentrations of oxycodone, hydrocodone, and fentanyl that were sampled from standards using the modular MasSpec Pen.

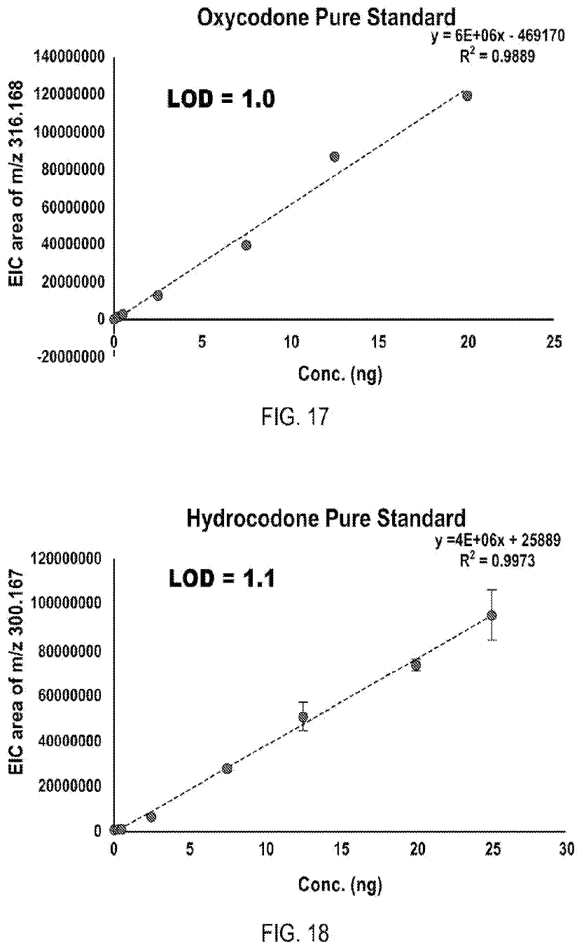

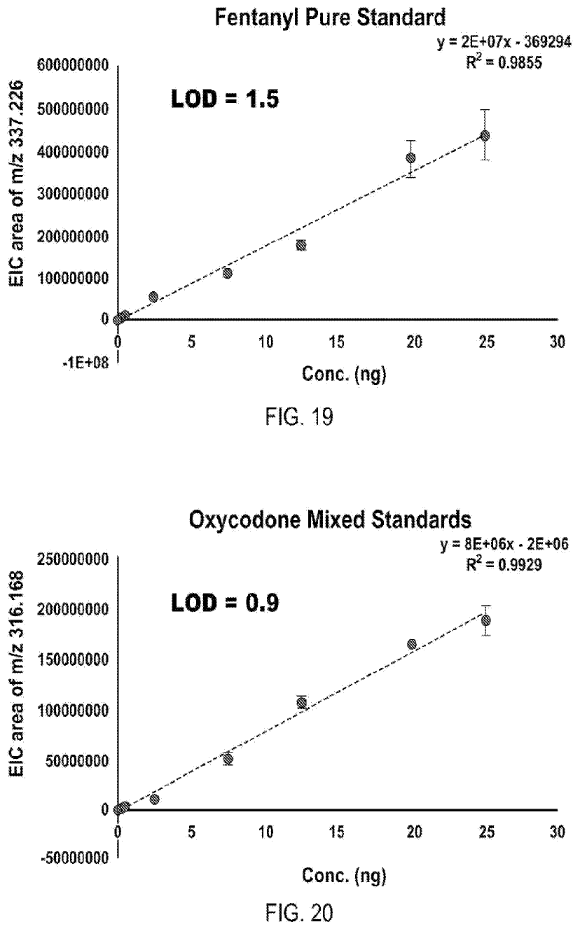

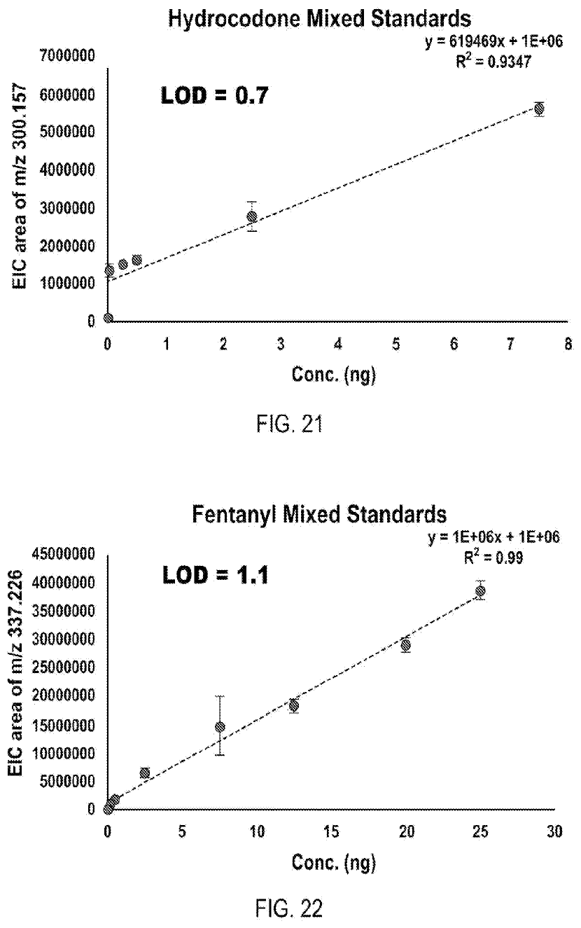

To evaluate the potential of the system for opioid screening, calibration curves were built for oxycodone, hydrocodone, and fentanyl sampled using the modular MasSpec Pen and analyzed by ESI (FIG. 17-FIG. 22). Calibration curves, generated using the average intensity of the precursor ion against concentration, yielded average LODs of 1.2±0.2 ng for oxycodone, 0.8±0.3 ng for hydrocodone, and 1.2±0.3ng for fentanyl. When sampling from a mixture of these drugs, average LODs of 1.2±0.3 ng, 0.6±0.1 ng, and 1.3±0.3 ng for oxycodone, hydrocodone, and fentanyl, respectively, were achieved. These initial results demonstrate that the modular MasSpec Pen allows sensitive sampling and further analysis of opioids from surfaces.

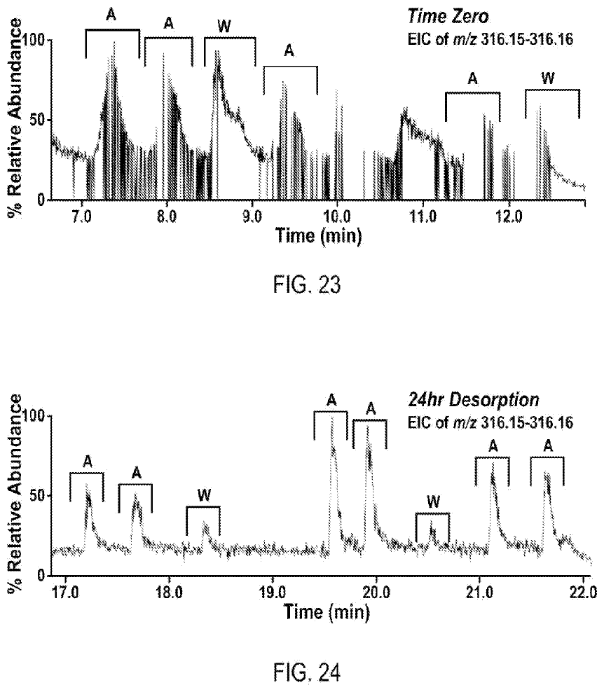

Oxycodone applied to the envisioned patient sample/biological matrix, human skin samples, was also analyzed. For the skin desorption studies, two solutions were made with different concentrations of oxycodone were made to use as the desorption solution, one having a concentration at Cₘₐₓ and the other around 0.5Cₘₐₓ (40 and 100 ppb). A previously developed high-throughput screening setup (Martins et al. International Journal of Pharmaceutics, 2019, 565, 557-568) was used to desorb the drug through the skin from dermis to epidermis. The epidermis was screened for oxycodone before and after the desorption. The EIC of oxycodone for time zero and after 24 hrs are shown in FIG. 23 and FIG. 24. The chronograms are quite salty due to the needed use of PBS as the desorption solution and to thaw the skin, but a signal can still be seen for oxycodone when evaluating the skin after desorption.

The performance and LODs for a panel of opioids is currently being evaluated.

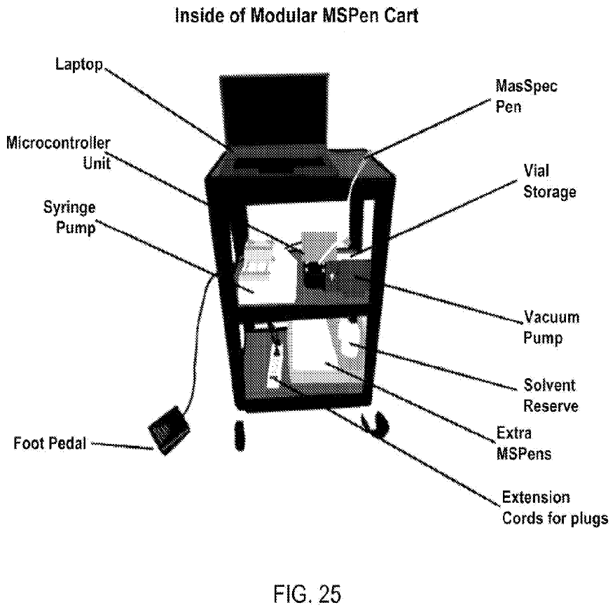

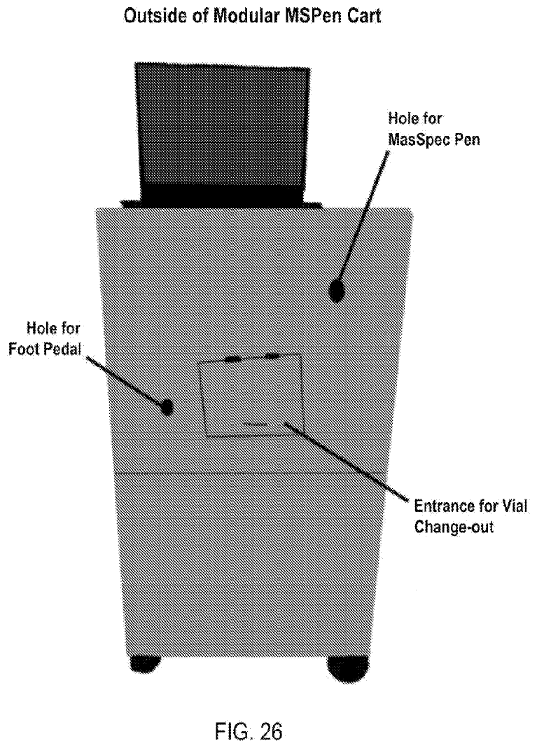

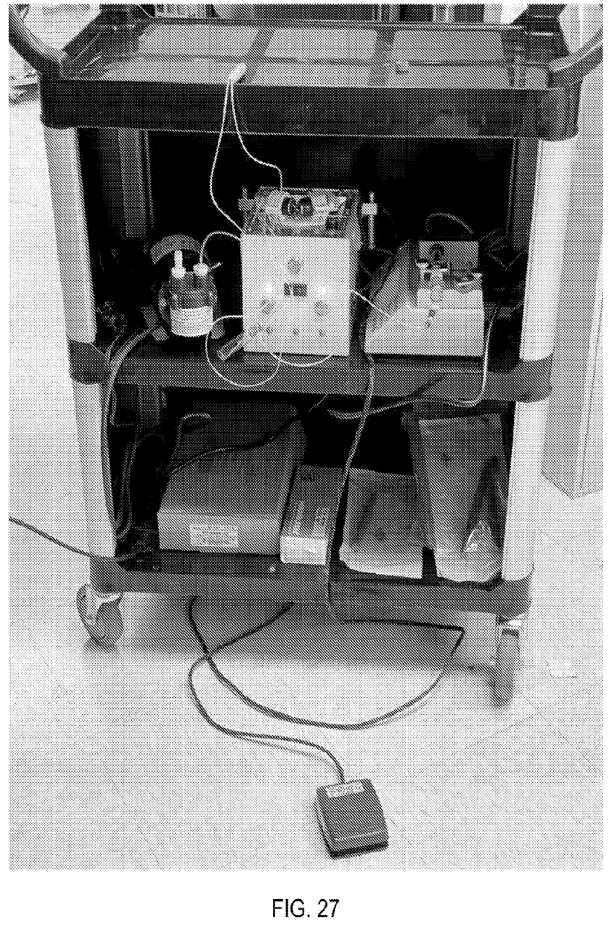

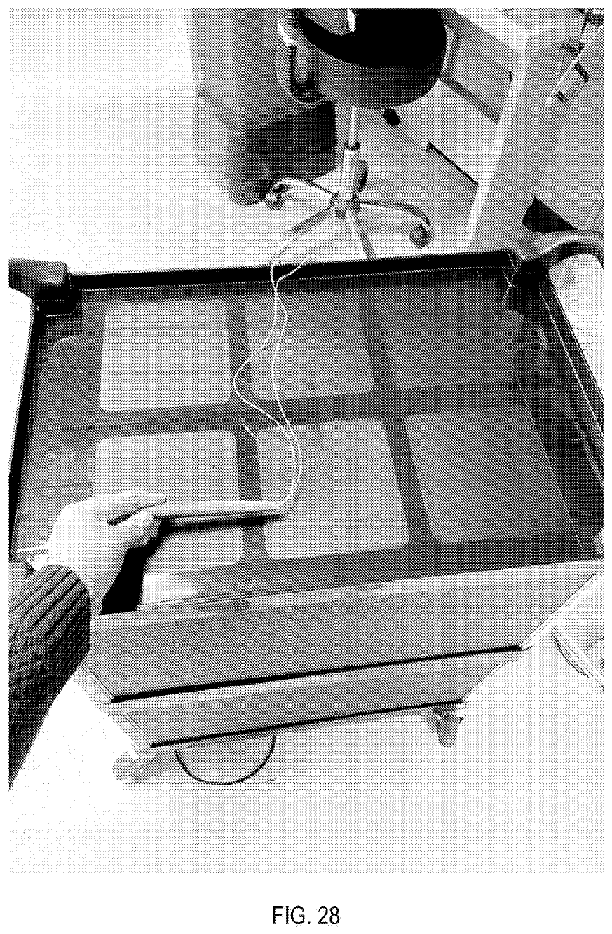

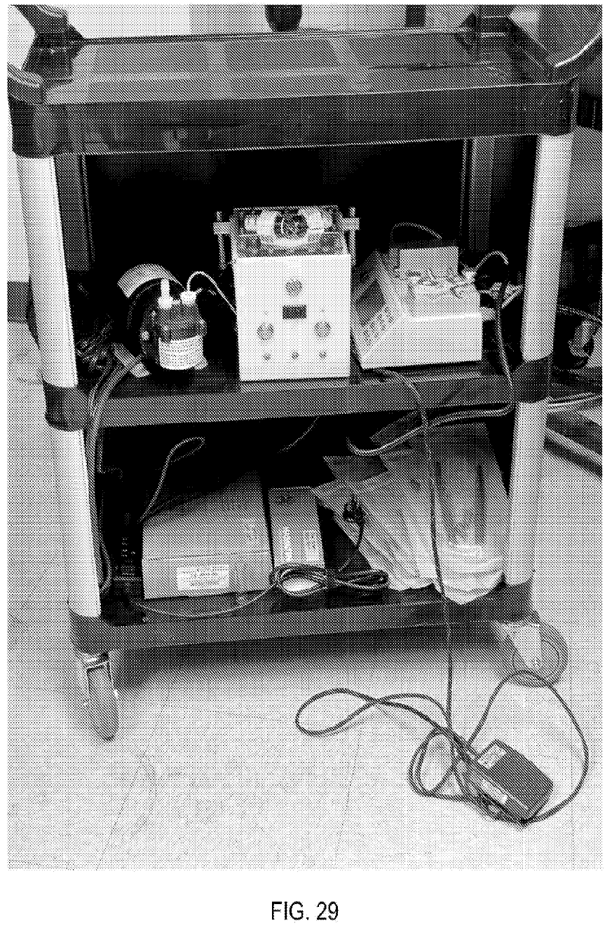

Further, a cart-like modular MasSpec pen was developed and the system is currently being tested to analyze patient samples and determine its applicability for monitoring patient drug use (FIG. 25-FIG. 29).

### Example 6—Detached MasSpec Pen

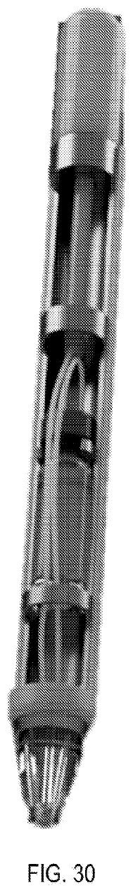

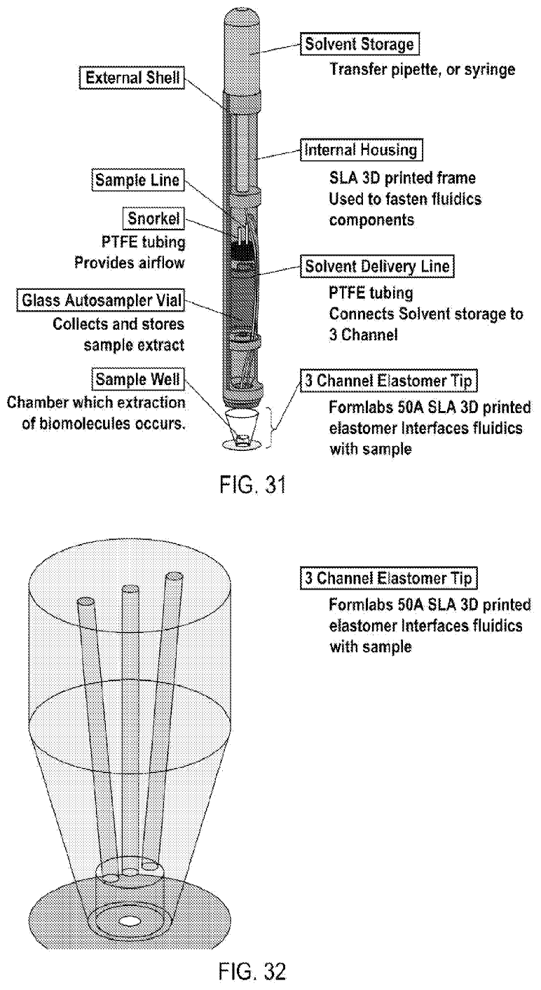

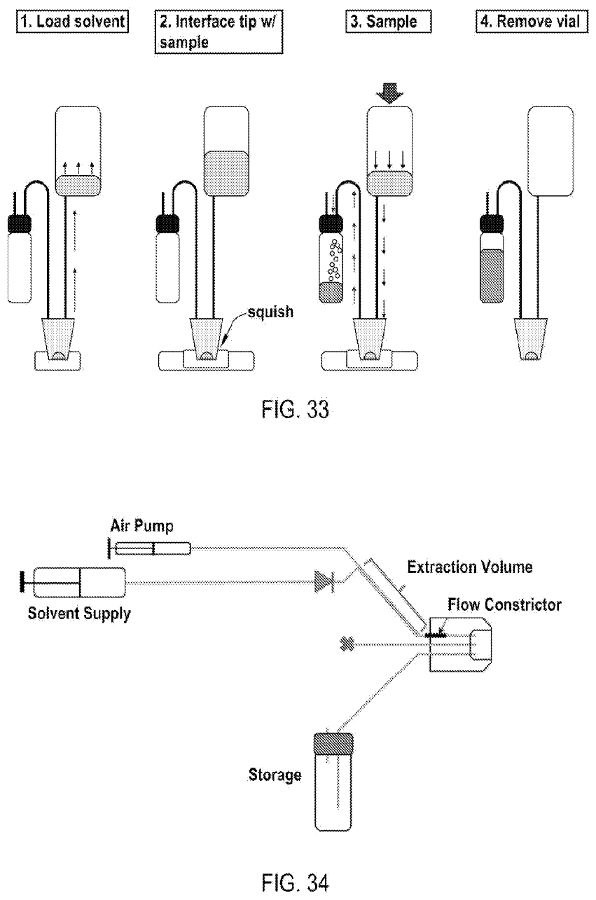

A concept design of a fully handheld, disposable, and/or autoclavable MasSpec Pen envisioned for intraoperative use is shown in FIG. 30-FIG. 31. The pictured device contains miniaturized fluidics that extract and store the sample within the device unit, with similar actuation to a laboratory pipette as described in FIG. 33. FIG. 32 is an enlarged schematic of the three channel elastomer tip.

### Example 7

In some examples, the device can further include a flow constrictor, as shown in FIG. 34. A detached MasSpec Pen (DMSP) fluidics circuit can deliver precise solvent volumes per extraction event.

Use of the flow constrictor can, for example, affect the content of the liquid sample. For example, using the flow constrictor can affect the presence and/or concentration of lipid(s) within the liquid sample.

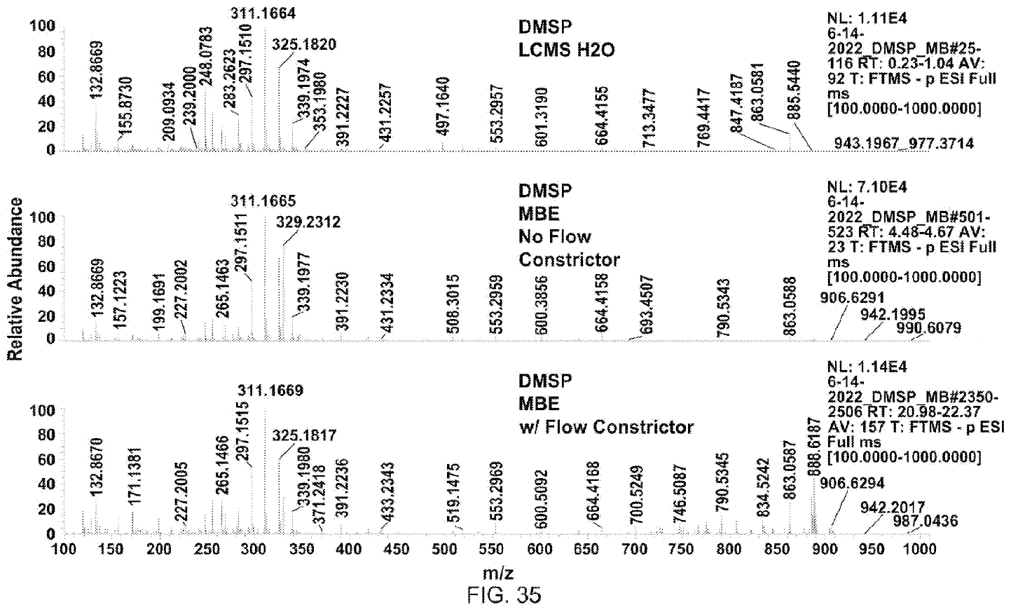

FIG. 35 illustrates the effect of flow constrictor on results collected for mouse brain extract (MBE) with detached MasSpec Pen (DMSP). The use of the flow constrictor increased the lipid signal (bottom trace).

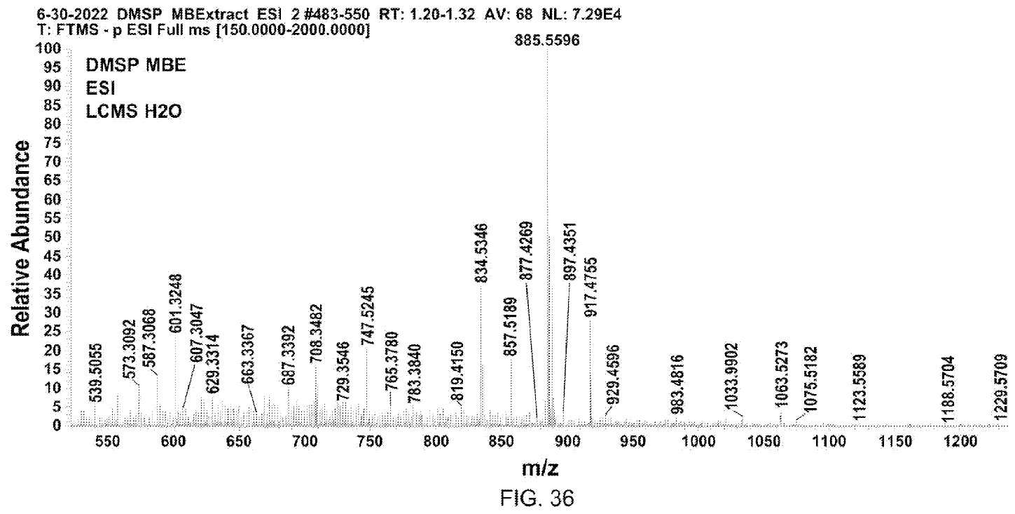

FIG. 36 shows LC-MS results for mouse brain extract (MBE) collected with detached MasSpec Pen (DMSP).

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

The methods of the appended claims are not limited in scope by the specific methods described herein, which are intended as illustrations of a few aspects of the claims and any methods that are functionally equivalent are intended to fall within the scope of the claims. Various modifications of the methods in addition to those shown and described herein are intended to fall within the scope of the appended claims. Further, while only certain representative method steps disclosed herein are specifically described, other combinations of the method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

1. An apparatus for producing a sample for analysis, the apparatus comprising:
a probe comprising a reservoir, a first conduit, a second conduit, and a third conduit;
wherein the reservoir is in fluid communication with the first conduit, the second conduit, and the third conduit; and
wherein, when the probe is assembled together with a chamber configured to contain a solvent, a gas supply, and a receptacle, then:
the first conduit is configured to be in fluid communication with the chamber, such that the first conduit is configured to deliver a discrete volume of the solvent to the reservoir;
the second conduit is configured to be in fluid communication with the gas supply, such that the gas supply is configured to deliver a gas to the reservoir; and
the third conduit is in fluid communication with the receptacle, such that the receptacle is configured to receive the sample from the reservoir.

2. The apparatus of claim 1, wherein the apparatus further comprises the receptacle, the chamber, the gas supply, or a combination thereof.

3. (canceled)

4. (canceled)

5. The apparatus of claim 1, the apparatus comprising:
the chamber configured to contain the solvent;
the gas supply;
the receptacle; and
the probe;
wherein:
the first conduit is in fluid communication with the chamber, such that the first conduit is configured to deliver a discrete volume of the solvent to the reservoir;
the second conduit is in fluid communication with the gas supply and the gas supply is configured to deliver the gas to the reservoir; and
the third conduit is in fluid communication with the receptacle and the receptacle is configured to receive the sample from the reservoir.

6. (canceled)

7. The apparatus of claim 1, wherein the solvent comprises water, an alcohol, acetonitrile, DMF, or a combination thereof.

8.-16. (canceled)

17. The apparatus of claim 1, wherein the gas comprises air, nitrogen, argon, carbon dioxide, or a combination thereof.

18. (canceled)

19. (canceled)

20. The apparatus of claim 1, wherein the gas supply is configured to provide the gas at atmospheric pressure.

21. The apparatus of claim 1, wherein the gas supply comprises the atmosphere around the apparatus.

22. The apparatus of claim 1, wherein the gas supply is a pressurized gas supply.

23.-29. (canceled)

30. The apparatus of claim 1, further comprising a pump in fluid communication with the chamber and the first conduit, wherein the pump is configured to transfer the discrete volume of the solvent from the chamber to the reservoir via the first conduit.

31. (canceled)

32. The apparatus of claim 1, wherein the discrete volume of the solvent is in direct contact with a surface, the surface being a sample site.

33. The apparatus of claim 32, wherein the solvent is delivered to the reservoir such that it contacts the sample site non-destructively.

34. (canceled)

35. (canceled)

36. The apparatus of claim 32, wherein the surface is at least a portion of a tissue from a subject.

37. The apparatus of claim 36, wherein the tissue is in vivo or ex vivo.

38.-45. (canceled)

46. The apparatus of claim 1, further comprising a waste container in fluid communication with the third conduit.

47. The apparatus of claim 46, further comprising a valve configured to diverge a fluid from the third conduit to the waste container.

48. (canceled)

49. (canceled)

50. The apparatus of claim 1, wherein the probe and/or the apparatus is configured to be hand-held.

51.-57. (canceled)

58. The apparatus of claim 1, further comprising a control system configured to control:
a solvent flow from the chamber through the first conduit to the reservoir;
a gas flow from the gas supply through the second conduit to the reservoir;
a sample flow from the reservoir through the third conduit to the receptacle;
or a combination thereof.

59. (canceled)

60. The apparatus of claim 58, wherein the control system comprises a haptic control device that controls solvent flow.

61. (canceled)

62. The apparatus of claim 1, wherein the apparatus is not directly coupled to an analyzer.

63. A method for collecting a sample from a surface using the apparatus of claim 1, the method comprising:
(a) contacting the probe with the surface;
(b) applying a fixed or discrete volume of the solvent to the surface;
(c) collecting the applied solvent to obtain a liquid sample; and
(d) storing the sample in the receptacle.

64.-111. (canceled)
